SEQUENCE LISTING (4 sequences)

SEQ ID NO: 1 - protein, 17 aa, ARTIFICIAL SEQUENCE
  synthetic polypeptide
ISQAVHAAHAEINEAGR

SEQ ID NO: 2 - protein, 23 aa, ARTIFICIAL SEQUENCE
  synthetic polypeptide
ATQLYKTCKQAGTCPPDIIPKVK

SEQ ID NO: 3 - protein, 23 aa, ARTIFICIAL SEQUENCE
  synthetic polypeptide
MSLLTEVETPTRNEWECRCSDGG

SEQ ID NO: 4 - protein, 23 aa, ARTIFICIAL SEQUENCE
  synthetic polypeptide
MSLLTEVETPIRNEWECRCSDGG

(19) United States
(12) Patent Application Publication
(10) Pub. No.: US 2012/0058153 A1
(43) Pub. Date: Mar. 8, 2012

(54) SYNTHETIC NANOCARRIER VACCINES COMPRISING PROTEINS OBTAINED OR DERIVED FROM HUMAN INFLUENZA A VIRUS HEMAGGLUTININ

(75) Inventors: Petr Ilyinskii, Cambridge, MA (US); Yun Gao, Southborough, MA (US); Grayson B. Lipford, Watertown, MA (US)

(73) Assignee: Selecta Biosciences, Inc., Watertown, MA (US)

(21) Appl. No.: 13/213,529

(22) Filed: Aug. 19, 2011

Related U.S. Application Data
(60) Provisional application No. 61/375,543, filed on Aug. 20, 2010.
(60) Provisional application No. 61/375,635, filed on Aug. 20, 2010.
(60) Provisional application No. 61/375,586, filed on Aug. 20, 2010.

(51) Int. Cl. A61K 39/385; A61K 9/14; A61P 31/16; A61P 37/04; C07K 14/11; C08G 65/333; B82Y 40/00; B82Y 5/00

(52) U.S. Cl. 424/400; 530/402; 525/54.1; 424/196.11; 977/906; 977/735; 977/762; 977/802; 977/797; 977/773; 977/832; 977/882

(57) ABSTRACT

This invention relates to compositions and methods that can be used immunize a subject against influenza. Generally, the compositions and methods include polypeptides obtained or derived from human influenza A virus hemagglutinin.

### RELATED APPLICATIONS

This application claims the benefit under 35 U.S.C. §119 of U.S. provisional applications 61/375,586, 61/375,635, and 61/375,543, each filed Aug. 20, 2010, the entire contents of each of which are incorporated herein by reference.

### FIELD OF THE INVENTION

This invention relates to compositions and methods that can be used immunize a subject against influenza. Generally, the compositions and methods include polypeptides obtained or derived from human influenza A virus hemagglutinin.

### BACKGROUND OF THE INVENTION

Influenza is an infectious disease caused by RNA viruses of the family Orthomyxoviridae. Common symptoms of the disease include chills, fever, sore throat, muscle pains, severe headache, coughing, and fatigue. In more serious cases, influenza can lead to pneumonia, which can be fatal. Influenza spreads around the world in seasonal epidemics, resulting in the deaths of between 250,000 and 500,000 people every year, and up to millions in some pandemic years. Human influenza A virus (“HIAV”) is the most common strain of the virus, and is responsible for all major influenza pandemics.

Major human influenza viral envelope component hemagglutinin (HA) is an influenza surface glycoprotein. HA is known to induce protective immune responses that can efficiently prevent viral infection and/or virus-induced disease in animal models and human subjects (Ellebedy and Webby, 2009; Roose et al., 2009).

HA exists in nature as a trimer composed of three identical monomers assembled into a central-helical coiled coil that consists of stem (stalk) region and three globular domains. The three globular domains contain binding sites for cell surface receptors that are essential for virus attachment to the target cell. The HA monomers are composed of two disulfide-linked glycoprotein chains, HA1 and HA2, which are created by proteolytic cleavage of the precursor HA0 during viral maturation. Aside from cell attachment, HA plays an essential role in infection by initiating a pH-dependent fusion of viral and endosomal membranes upon endocytosis. This fusion process induces dramatic conformational changes in HA2, which involve translocations of several HA2 domains, exposure of fusion peptides and formation of hydrophobic bonds between HA and the target membrane (Cross et al. 2009; Isin et al., 2002; Ekiert et al., 2009).

HA is known to induce strong antibody-mediated immune responses against influenza virus and is a central component of many influenza vaccines. However, utilization of HA-based vaccines is plagued with two well-known problems. These inherent issues undercutting HA-based immunization schemes are closely related to HIAV biology. HIAV possesses an ability to constantly acquire new structural mutations and thus change antigenically (antigenic drift). HIAV also possesses a capacity for gene exchange and recombination, which often results in generation of a viral strain with a completely novel surface gene composition (antigenic shift).

Continuous antigenic changes of HIAV necessitate seasonal construction of influenza vaccine de novo utilizing those HA protein that are carried by the viral strains predicted to cause epidemics during the next season. This approach is prone to mistakes as during 2007-2008 epidemics, when two of the three vaccines prepared early in the year failed to target those viral strains that actually emerged. Moreover, it requires repeated manufacturing high amounts of vaccine containing different HAs, which sometimes (as during the most recent 2009-2010 season) may not be accomplished timely to provide sufficient vaccination material for the general population.

Constant changes in HA leads to accumulation of mutations in its dominant antigenic epitopes, which manifestly contributes to the non-stop waning of anti-HA immunity in a vaccinated population. These epitopes are mostly localized in HA variable globular regions, which are easily accessible to antibodies and are being targeted by humoral response in a majority of vaccinated individuals or animals (Caton et al., 1982; Kaverin et al., 2002; Tsuchiya et al., 2001; Wiley et al. 1981). Thus, HAs of new viral strains continuously emerging by antigenic drift are recognized less efficiently, which leads to a constant decrease of protection in a vaccinated population. Moreover, current HIAV vaccines won't protect against already existing viral strains that carry HA types unrelated to those used for vaccination. Furthermore, HA-directed immunity may be essentially ineffective against a completely novel HIAV strain, emerging as a result of antigenic shift.

### SUMMARY OF THE INVENTION

In one aspect, a dosage form comprising synthetic nanocarriers that are coupled to polypeptides obtained or derived from human influenza A virus hemagglutinin is provided. In one embodiment, the polypeptides are glycosylated. In another embodiment, the polypeptides comprise an entire human influenza A virus hemagglutinin. In still another embodiment, the polypeptides comprise a fragment of human influenza A virus hemagglutinin. In a further embodiment, the polypeptides are obtained or derived from an HA1 subunit of human influenza A virus hemagglutinin. In still a further embodiment, the polypeptides comprise an entire HA1 subunit of human influenza A virus hemagglutinin. In yet a further embodiment, the polypeptides comprise a fragment of HA1 subunit of human influenza A virus hemagglutinin. In still another embodiment, the polypeptides are obtained or derived from an HA2 subunit of human influenza A virus hemagglutinin. In yet another embodiment, the polypeptides comprise an entire HA2 subunit of human influenza A virus hemagglutinin. In one embodiment, the polypeptides comprise a fragment of HA2 subunit of human influenza A virus hemagglutinin. In another embodiment, the polypeptides comprise any of the polypeptides provided herein. In some embodiments, the polypeptides coupled to the synthetic nanocarriers are of the same type (i.e., are identical). In other embodiments, two or more types of polypeptides are coupled to the synthetic nanocarriers.

In another embodiment, the synthetic nanocarriers are further coupled to one or more adjuvants. In one embodiment, the one or more adjuvants comprise Pluronic® block co-polymers, specifically modified or prepared peptides, stimulators or agonists of pattern recognition receptors, mineral salts, alum, alum combined with monphosphoryl lipid (MPL) A of Enterobacteria, MPL® (AS04), saponins, QS-21, Quil-A, ISCOMs, ISCOMATRIXT™, MF59™, Montanide® ISA 51, Montanide® ISA 720, AS02, liposomes and liposomal formulations, AS01, synthesized or specifically prepared microparticles and microcarriers, bacteria-derived outer membrane vesicles of N. gonorrheae or Chlamydia trachomatis, chitosan particles, depot-forming agents, muramyl dipeptide, aminoalkyl glucosaminide 4-phosphates, RC529, bacterial toxoids, toxin fragments, agonists of Toll-Like Receptors 2, 3, 4, 5, 7, 8, 9 and/or combinations thereof; adenine derivatives; immunostimulatory DNA; immunostimulatory RNA; imidazoquinoline amines, imidazopyridine amines, 6,7-fused cycloalkylimidazopyridine amines, 1,2-bridged imidazoquinoline amines; imiquimod; resiquimod; type I interferons; bacterial lipopolysacccharide (LPS); VSV-G; HMGB-1; flagellin or portions or derivatives thereof; or immunostimulatory DNA molecules comprising CpGs, agonists for DC surface molecule CD40; type I interferons; poly I:C; poly I:C12U; bacterial lipopolysacccharide (LPS); VSV-G; HMGB-1; flagellin or portions or derivatives thereof; immunostimulatory DNA molecules comprising CpGs; proinflammatory stimuli released from necrotic cells; urate crystals; activated components of the complement cascade; activated components of immune complexes; complement receptor agonists; cytokines; or cytokine receptor agonists. In another embodiment, the one or more adjuvants comprise agonists of Toll-Like Receptors 2, 3, 4, 5, 7, 8, 9 and/or combinations thereof; adenine derivatives; immunostimulatory DNA; immunostimulatory RNA; imidazoquinoline amines, imidazopyridine amines, 6,7-fused cycloalkylimidazopyridine amines, 1,2-bridged imidazoquinoline amines, imiquimod, resiquimod, immunostimulatory DNA molecules comprising CpGs, poly I:C or poly I:C12U.

In one embodiment, the synthetic nanocarriers comprise lipid-based nanoparticles, polymeric nanoparticles, metallic nanoparticles, surfactant-based emulsions, dendrimers, buckyballs, nanowires, virus-like particles, peptide or protein-based particles, lipid-polymer nanoparticles, spheroidal nanoparticles, cubic nanoparticles, pyramidal nanoparticles, oblong nanoparticles, cylindrical nanoparticles, or toroidal nanoparticles. In another embodiment, the synthetic nanocarriers comprise poly(lactic acid)-polyethyleneglycol copolymer, poly(glycolic acid)-polyethyleneglycol copolymer, or poly(lactic-co-glycolic acid)-polyethyleneglycol copolymer.

In yet another embodiment, the synthetic nanocarriers are further coupled to one or more T-helper antigens. In one embodiment, the T-helper antigen comprises any of the T-helper antigens provided herein. In another embodiment, the amino acid sequence of the T-helper antigen comprises the amino acid sequence as set forth in SEQ ID NO: 1.

In a further embodiment, the synthetic nanocarriers are present in an amount effective to provide an immune response to the polypeptides when the dosage form is administered to a subject.

In yet a further embodiment, the dosage form further comprises influenza antigen that is not coupled to the synthetic nanocarriers.

In one embodiment, at least a portion of the polypeptides obtained or derived from human influenza A virus hemagglutinin are coupled to a surface of the synthetic nanocarriers. In another embodiment, the synthetic nanocarriers are covalently coupled to polypeptides obtained or derived from human influenza A virus hemagglutinin. In yet another embodiment, the synthetic nanocarriers are non-covalently coupled to polypeptides obtained or derived from human influenza A virus hemagglutinin.

In one embodiment, the dosage form further comprises a pharmaceutically acceptable excipient.

In another aspect, a method comprising administering any of the dosage forms provided to a subject is provided. In one embodiment, the dosage form is administered at least once to the subject. In another embodiment, the dosage form is administered at least twice to the subject. In still another embodiment, the dosage form is administered at least three times to the subject. In yet another embodiment, the dosage form is administered at least four times to the subject.

In yet another aspect, a method comprising providing synthetic nanocarriers, and coupling polypeptides that are obtained or derived from human influenza A virus hemagglutinin to the synthetic nanocarriers is provided. The polypeptides may be any of the polypeptides provided herein. In some embodiments, the polypeptides coupled to the synthetic nanocarriers are of the same type (i.e., are identical). In other embodiments, two or more types of polypeptides are coupled to the synthetic nanocarriers. In one embodiment, the coupling comprises covalently coupling the polypeptides to the synthetic nanocarriers.

In still another aspect, a composition, dosage form or vaccine obtained, or obtainable, by any of the methods provided herein is provided.

In yet another aspect, a process for producing a composition, dosage form or vaccine comprising the steps of providing synthetic nanocarriers, and coupling polypeptides that are obtained or derived from human influenza A virus hemagglutinin to the synthetic nanocarriers is provided. Again, the polypeptides may be any of the polypeptides provided herein. In some embodiments, the polypeptides coupled to the synthetic nanocarriers are of the same type (i.e., are identical). In other embodiments, two or more types of polypeptides are coupled to the synthetic nanocarriers.

In still another aspect, any of the dosage forms provided may be for use in therapy or prophylaxis. In yet another aspect, any of the dosage forms provided may be for use in any of the methods provided. In a further aspect, any of the dosage forms provided may be for use in vaccination. In yet a further aspect, any of the dosage forms provided may be for use in a method of therapy or prophylaxis of influenza virus infection, for example, influenza A virus infection. In yet another aspect, any of the dosage forms provided may be for use in a method of therapy or prophylaxis comprising administration by a subcutaneous, intramuscular, intradermal, oral, intranasal, transmucosal, sublingual, rectal, ophthalmic, transdermal, transcutaneous route or by a combination of these routes. In still another aspect, a use of any of the dosage forms provided for the manufacture of a medicament, for example a vaccine, for use in any of the methods provided is provided.

### BRIEF DESCRIPTION OF THE DRAWINGS

FIG. 1 shows the titres from HA vaccination. Group 1: nanocarrier-HA protein conjugates (NC-HA), group 2: immunized with 1 μg of HA protein; group 3: immunized with 1 μg of HA in Imject alum (Thermo Scientific, w/w=1:1).

FIG. 2 shows the titres from HA vaccination using NC-HA in the presence of nanocarriers containing other proteins or peptides. Group 1: immunized with nanocarrier-HA protein conjugates (NC-HA) and nanocarrier-ovalbumin protein conjugates (NC-OVA). Group 2: immunized with NC-HA, NC-OVA, and nanocarrier-M2e peptide-L2 peptide conjugates (NC-M2e-L2; influenza M2e peptide, HPV L2 peptide).

### DETAILED DESCRIPTION OF THE INVENTION

### I. Introduction

Significant investment of public and medical efforts is necessary for targeting HIAV, encompassing constant epidemiologic surveillance of influenza and annual re-vaccination of susceptible populations with vaccines carrying different HA subtypes. At the same time, production of vaccines against novel strains takes time and these are often delivered to general public with a considerable delay, which may result in significant morbidity and mortality if a highly pathogenic and unique influenza strain emerges in the future. Therefore, any improvement of an influenza vaccine that will provide for antigen sparing or faster or easier means of vaccine manufacturing that will result in much speedier delivery to the general population of a vaccine protecting against novel viral strains will have a significant public health importance. Therefore, what is needed are compositions and methods that could address the problems noted above that are associated with producing vaccines against human influenza A virus.

The inventors have unexpectedly and surprisingly discovered that the problems and limitations noted above can be overcome by practicing the invention disclosed herein. In particular, the inventors have unexpectedly discovered that it is possible to provide dosage forms, and related methods, that comprise synthetic nanocarriers that are coupled to polypeptides obtained or derived from human influenza A virus hemagglutinin. The inventors have further discovered that it is possible to provide methods comprising: providing synthetic nanocarriers; and coupling polypeptides that are obtained or derived from human influenza A virus hemagglutinin to the synthetic nanocarriers.

HA is known to induce strong antibody-mediated immune response against human influenza A virus and is a central component of many influenza vaccines. As noted above, conventional approaches to generation of a vaccine against human influenza A virus hemagglutinin utilize either the entire glycoprotein or a fragment thereof. The present invention provides for a vaccine based on synthetic nanocarriers coupled to human influenza A virus hemagglutinin, including the entire glycoprotein or a fragment thereof. This approach facilitates the utilization of smaller quantities of HA protein for influenza immunization thus enabling so-called antigen sparing which is important for timely vaccine delivery especially if ongoing epidemic is induced by novel and/or highly-pathogenic influenza strain. Furthermore, this approach enables the utilization of recombinant HA (compared to virally-produced) coupled to NC, which will further shorten the time of vaccine production response to an emergence of a novel pandemic strain.

The Examples below illustrate a coupling of several viral antigens to polymeric synthetic PLA/PLGA-based nanocarrier (NC) via PLA-PEG linker. These antigens include HA glycoprotein from a highly pathogenic strain A/Vietnam/1203/04(H5N1), a.k.a. “avian flu”. These Examples provide experimental evidence demonstrating that coupling HA to synthetic nanocarriers permits use of similar or lower HA quantities, as compared to conventional HA-based vaccines, to attain markedly higher immunogenicity than generated by the conventional HA-based vaccines (i.e. antigen sparing).

Example 1 illustrates an embodiment wherein HA polypeptides containing suitable linkers are conjugated to synthetic nanocarriers via click type chemistry such as Copper-catalyzed azide-alkyne cycloaddition reaction. Examples 2 and 3 illustrate embodiments wherein HA polypeptides can also be conjugated to other synthetic carriers via non-covalent bonding such as ionic interaction or conjugated to virus-like-particles such as RNA bacteriophages, cowpea mosaic virus, tobacco mosaic virus, etc.

In Examples 4 and 5, the HA used (H5A/Vietnam/1203/2004) has a MW of 72 K. About 2 mg (3×10-5 mmol) of the protein was used in the NC coupling via EDC/NHS conjugation using ca. 14 mg of NCs (containing ca. 3 mg of PLA-PEG-CO2H, 1×10-4 mmol) with a 100% theoretical coupling efficiency resulting in loads of 125 μg of HA per 1 mg of NC (2 mg HA/16 mg total NC-HA mass). The actual efficiency of EDC/NHS protein coupling to NC is known to be in the range of 1-20% (Thorek, D. L. J., Elias, D. R., Tsourkas, A. Comparative Analysis of Nanoparticle-Antibody Conjugations: Carbodiimide versus Click Chemistry. Molecular Imaging, 2009, 8(4):221-229). Accordingly, the actual load of NC-coupled HA is estimated to be 1.25-25 μg HA per 1 mg of NC.

Thus, 100 μg of HA-carrying NC as used for immunization in Example 5 are estimated to carry 0.125-2.5 μg of HA, which is approximately the same (or, possibly, lower) protein quantity as was used for immunization with purified HA (1 μg). Using 100 μg of such HA-coupled NC, it was possible to generate antibody titers that were 50 times higher than those induced by 1 μg of purified HA protein or 5 times higher than those induced by 1 μg of HA protein admixed with commercially used alum adjuvant (Example 5). Collectively, by employing this novel paradigm, the present invention provides uniquely efficient HA-carrying immunogens, which permits much more efficient utilization of HA than currently used vaccines (antigen sparing).

Before describing the present invention in detail, it is to be understood that this invention is not limited to particularly exemplified materials or process parameters as such may, of course, vary. It is also to be understood that the terminology used herein is for the purpose of describing particular embodiments of the invention only, and is not intended to be limiting of the use of alternative terminology to describe the present invention.

All publications, patents and patent applications cited herein, whether supra or infra, are hereby incorporated by reference in their entirety for all purposes.

As used in this specification and the appended claims, the singular forms “a,” “an” and “the” include plural referents unless the content clearly dictates otherwise. For example, reference to “a polymer” includes a mixture of two or more such molecules or a mixture of differing molecular weights of a single polymer species, reference to “a synthetic nanocarrier” includes a mixture of two or more such synthetic nanocarriers or a plurality of such synthetic nanocarriers, reference to a “DNA molecule” includes a mixture of two or more such DNA molecules or a plurality of such DNA molecules, reference to “an adjuvant” includes a mixture of two or more such materials or a plurality of adjuvant molecules, and the like.

As used herein, the term “comprise” or variations thereof such as “comprises” or “comprising” are to be read to indicate the inclusion of any recited integer (e.g. a feature, element, characteristic, property, method/process step or limitation) or group of integers (e.g. features, element, characteristics, properties, method/process steps or limitations) but not the exclusion of any other integer or group of integers. Thus, as used herein, the term “comprising” is inclusive and does not exclude additional, unrecited integers or method/process steps.

In embodiments of any of the compositions and methods provided herein, “comprising” may be replaced with “consisting essentially of” or “consisting of”. The phrase “consisting essentially of” is used herein to require the specified integer(s) or steps as well as those which do not materially affect the character or function of the claimed invention. As used herein, the term “consisting” is used to indicate the presence of the recited integer (e.g. a feature, element, characteristic, property, method/process step or limitation) or group of integers (e.g. features, element, characteristics, properties, method/process steps or limitations) alone.

The invention will be described in more detail below.

### B. Definitions

“Adjuvant” means an agent that does not constitute a specific antigen, but boosts the strength and longevity of immune response to a concomitantly administered antigen. Such adjuvants may include, but are not limited to stimulators of pattern recognition receptors, such as Toll-like receptors, RIG-1 and NOD-like receptors (NLR), mineral salts, such as alum, alum combined with monphosphoryl lipid (MPL) A of Enterobacteria, such as Escherichia coli, Salmonella minnesota, Salmonella typhimurium, or Shigella flexneri or specifically with MPL® (AS04), MPL A of above-mentioned bacteria separately, saponins, such as QS-21, Quil-A, ISCOMs, ISCOMATRIX™, emulsions such as MF59™, Montanide® ISA 51 and ISA 720, AS02 (QS21+squalene+MPL®), liposomes and liposomal formulations such as AS01, synthesized or specifically prepared microparticles and microcarriers such as bacteria-derived outer membrane vesicles (OMV) of N. gonorrheae, Chlamydia trachomatis and others, or chitosan particles, depot-forming agents, such as Pluronic® block co-polymers, specifically modified or prepared peptides, such as muramyl dipeptide, aminoalkyl glucosaminide 4-phosphates, such as RC529, or proteins, such as bacterial toxoids or toxin fragments.

In embodiments, adjuvants comprise agonists for pattern recognition receptors (PRR), including, but not limited to Toll-Like Receptors (TLRs), specifically TLRs 2, 3, 4, 5, 7, 8, 9 and/or combinations thereof. In other embodiments, adjuvants comprise agonists for Toll-Like Receptors 3, agonists for Toll-Like Receptors 7 and 8, or agonists for Toll-Like Receptor 9; preferably the recited adjuvants comprise imidazoquinolines; such as R848 (also known as resiquimod); adenine derivatives, such as those disclosed in U.S. Pat. No. 6,329,381 (Sumitomo Pharmaceutical Company); US Published Patent Application 2010/0075995 to Biggadike et al., or WO 2010/018132 to Campos et al.; immunostimulatory DNA; or immunostimulatory RNA.

In specific embodiments, synthetic nanocarriers incorporate as adjuvants compounds that are agonists for toll-like receptors (TLRs) 7 & 8 (“TLR 7/8 agonists”). Of utility are the TLR 7/8 agonist compounds disclosed in U.S. Pat. No. 6,696,076 to Tomai et al., including but not limited to imidazoquinoline amines, imidazopyridine amines, 6,7-fused cycloalkylimidazopyridine amines, and 1,2-bridged imidazoquinoline amines. Preferred adjuvants comprise imiquimod and resiquimod (R848). In specific embodiments, an adjuvant may be an agonist for the DC surface molecule CD40. In certain embodiments, to stimulate immunity rather than tolerance, a synthetic nanocarrier incorporates an adjuvant that promotes DC maturation (needed for priming of naive T cells) and the production of cytokines, such as type I interferons, which promote antibody immune responses.

In embodiments, adjuvants also may comprise immunostimulatory RNA molecules, such as but not limited to dsRNA, poly I:C or poly I:poly C12U (available as Ampligen®, both poly I:C and poly I:poly C12U being known as TLR3 stimulants), and/or those disclosed in F. Heil et al., “Species-Specific Recognition of Single-Stranded RNA via Toll-like Receptor 7 and 8” Science 303(5663), 1526-1529 (2004); J. Vollmer et al., “Immune modulation by chemically modified ribonucleosides and oligoribonucleotides” WO 2008033432 A2; A. Forsbach et al., “Immunostimulatory oligoribonucleotides containing specific sequence motif(s) and targeting the Toll-like receptor 8 pathway” WO 2007062107 A2; E. Uhlmann et al., “Modified oligoribonucleotide analogs with enhanced immunostimulatory activity” U.S. Pat. Appl. Publ. US 2006241076; G. Lipford et al., “Immunostimulatory viral RNA oligonucleotides and use for treating cancer and infections” WO 2005097993 A2; G. Lipford et al., “Immunostimulatory G,U-containing oligoribonucleotides, compositions, and screening methods” WO 2003086280 A2. In some embodiments, an adjuvant may be a TLR-4 agonist, such as bacterial lipopolysacccharide (LPS), VSV-G, and/or HMGB-1. In some embodiments, adjuvants may comprise TLR-5 agonists, such as flagellin, or portions or derivatives thereof, including but not limited to those disclosed in U.S. Pat. Nos. 6,130,082, 6,585,980, and 7,192,725.

In specific embodiments, synthetic nanocarriers incorporate a ligand for Toll-like receptor (TLR)-9, such as immunostimulatory DNA molecules comprising CpGs, which induce type I interferon secretion, and stimulate T and B cell activation leading to increased antibody production and cytotoxic T cell responses (Krieg et al., CpG motifs in bacterial DNA trigger direct B cell activation. Nature. 1995. 374:546-549; Chu et al. CpG oligodeoxynucleotides act as adjuvants that switch on T helper 1 (Th1) immunity. J. Exp. Med. 1997. 186:1623-1631; Lipford et al. CpG-containing synthetic oligonucleotides promote B and cytotoxic T cell responses to protein antigen: a new class of vaccine adjuvants. Eur. J. Immunol. 1997. 27:2340-2344; Roman et al. Immunostimulatory DNA sequences function as T helper-1-promoting adjuvants. Nat. Med. 1997. 3:849-854; Davis et al. CpG DNA is a potent enhancer of specific immunity in mice immunized with recombinant hepatitis B surface antigen. J. Immunol. 1998. 160:870-876; Lipford et al., Bacterial DNA as immune cell activator. Trends Microbiol. 1998. 6:496-500; U.S. Pat. No. 6,207,646 to Krieg et al.; U.S. Pat. No. 7,223,398 to Tuck et al.; U.S. Pat. No. 7,250,403 to Van Nest et al.; or U.S. Pat. No. 7,566,703 to Krieg et al.

In some embodiments, adjuvants may be proinflammatory stimuli released from necrotic cells (e.g., urate crystals). In some embodiments, adjuvants may be activated components of the complement cascade (e.g., CD21, CD35, etc.). In some embodiments, adjuvants may be activated components of immune complexes. The adjuvants also include complement receptor agonists, such as a molecule that binds to CD21 or CD35. In some embodiments, the complement receptor agonist induces endogenous complement opsonization of the synthetic nanocarrier. In some embodiments, adjuvants are cytokines, which are small proteins or biological factors (in the range of 5 kD-20 kD) that are released by cells and have specific effects on cell-cell interaction, communication and behavior of other cells. In some embodiments, the cytokine receptor agonist is a small molecule, antibody, fusion protein, or aptamer.

In embodiments, at least a portion of the dose of adjuvant may be coupled to synthetic nanocarriers, preferably, all of the dose of adjuvant is coupled to synthetic nanocarriers. In other embodiments, at least a portion of the dose of the adjuvant is not coupled to the synthetic nanocarriers. In embodiments, the dose of adjuvant comprises two or more types of adjuvants. For instance, and without limitation, adjuvants that act on different TLR receptors may be combined. As an example, in an embodiment a TLR 7/8 agonist may be combined with a TLR 9 agonist. In another embodiment, a TLR 7/8 agonist may be combined with a TLR 4 agonist. In yet another embodiment, a TLR 9 agonist may be combined with a TLR 3 agonist.

“Administering” or “administration” means providing a drug to a subject in a manner that is pharmacologically useful.

“Amount effective” is any amount of a composition provided herein that produces one or more desired immune responses. This amount can be for in vitro or in vivo purposes. For in vivo purposes, the amount can be one that a clinician would believe may have a clinical benefit for a subject at risk of contracting an influenza infection, e.g., a human influenza A virus infection. Amounts effective include amounts that generate a humoral and/or cytotoxic T lymphocyte immune response, or certain levels thereof. An amount that is effective to produce a desired immune responses as provided herein can also be an amount that produces a desired therapeutic endpoint or a desired therapeutic result (e.g., prevents or reduces the severity of influenza infection in a subject).

A subject's immune response can be monitored by routine methods. Amounts effective will depend, of course, on the particular subject being treated; the severity of a condition, disease or disorder; the individual patient parameters including age, physical condition, size and weight; the duration of the treatment; the nature of concurrent therapy (if any); the specific route of administration and like factors within the knowledge and expertise of the health practitioner. These factors are well known to those of ordinary skill in the art and can be addressed with no more than routine experimentation. It is generally preferred that a maximum dose be used, that is, the highest safe dose according to sound medical judgment. It will be understood by those of ordinary skill in the art, however, that a patient may insist upon a lower dose or tolerable dose for medical reasons, psychological reasons or for virtually any other reasons.

“Antigen” means a B cell antigen or T cell antigen. In embodiments, antigens are coupled to the synthetic nanocarriers. In other embodiments, antigens are not coupled to the synthetic nanocarriers. In embodiments antigens are coadministered with the synthetic nanocarriers. In other embodiments antigens are not coadministered with the synthetic nanocarriers. “Type(s) of antigens” means molecules that share the same, or substantially the same, antigenic characteristics.

“At least a portion of the dose” means at least some part of the dose, ranging up to including all of the dose.

“B cell antigen” means any antigen that is or recognized by and triggers an immune response in a B cell (e.g., an antigen that is specifically recognized by a B cell receptor on a B cell). In some embodiments, an antigen that is a T cell antigen is also a B cell antigen. In other embodiments, the T cell antigen is not also a B cell antigen.

“Couple” or “Coupled” or “Couples” (and the like) means to chemically associate one entity (for example a moiety) with another. In some embodiments, the coupling is covalent, meaning that the coupling occurs in the context of the presence of a covalent bond between the two entities. In non-covalent embodiments, the non-covalent coupling is mediated by non-covalent interactions including but not limited to charge interactions, affinity interactions, metal coordination, physical adsorption, host-guest interactions, hydrophobic interactions, TT stacking interactions, hydrogen bonding interactions, van der Waals interactions, magnetic interactions, electrostatic interactions, dipole-dipole interactions, and/or combinations thereof. In embodiments, encapsulation is a form of coupling.

“Concomitantly” means administering two or substances to a subject in a manner that is correlated in time, preferably sufficiently correlated in time so as to provide a modulation in an immune response. In embodiments, concomitant administration may occur through administration of two or more substances in the same dosage form. In other embodiments, concomitant administration may encompass administration of two or more substances in different dosage forms, but within a specified period of time, preferably within 1 month, more preferably within 1 week, still more preferably within 1 day, and even more preferably within 1 hour.

“Derived” means taken from a source and subjected to substantial modification. For instance, a polypeptide or nucleic acid with a sequence with only 50% identity to a natural polypeptide or nucleic acid, preferably a natural consensus polypeptide or nucleic acid, would be said to be derived from the natural polypeptide or nucleic acid. Substantial modification is modification that significantly affects the chemical or immunological properties of the material in question. Derived polypeptides and nucleic acids can also include those with a sequence with greater than 50% identity to a natural polypeptide or nucleic acid sequence if said derived polypeptides and nucleic acids have altered chemical or immunological properties as compared to the natural polypeptide or nucleic acid. These chemical or immunological properties comprise hydrophilicity, stability, affinity, and ability to couple with a carrier such as a synthetic nanocarrier.

“Dosage form” means a pharmacologically and/or immunologically active material in a medium, carrier, vehicle, or device suitable for administration to a subject.

“Encapsulate” means to enclose at least a portion of a substance within a synthetic nanocarrier. In some embodiments, a substance is enclosed completely within a synthetic nanocarrier. In other embodiments, most or all of a substance that is encapsulated is not exposed to the local environment external to the synthetic nanocarrier. In other embodiments, no more than 50%, 40%, 30%, 20%, 10% or 5% is exposed to the local environment. Encapsulation is distinct from absorption, which places most or all of a substance on a surface of a synthetic nanocarrier, and leaves the substance exposed to the local environment external to the synthetic nanocarrier.

“Entire” means that greater than 75% of the consensus amino acid sequence of a polypeptide is present, taken as an average across a sample of the material. In embodiments, greater than 80%, greater than 85%, greater than 90%, or greater than 95%, of the consensus amino acid sequence of a polypeptide is present, taken as an average across a sample of the material. In general the amount of the consensus sequence that is present can be determined using conventional methods. In one embodiment, GPC-HPLC (gel permeation chromatography-high pressure liquid chromatography) could be used for determining the molecular weight of the glycosylated polypeptide, and then the Lowry assay and a phenol-sulfuric acid assay could be used to determine the amount of amino acid and saccharide material, respectively.

“Fragment” means that less than 75% of the consensus sequence of a polypeptide is present, taken as an average across a sample of the material. In embodiments, less than 70%, less than 65%, less than 60%, less than 55%, or less than 50%, of the consensus sequence of a polypeptide is present, taken as an average across a sample of the material. In general the amount of the consensus sequence that is present can be determined using conventional methods. In one embodiment, GPC—HPLC (gel permeation chromatography-high pressure liquid chromatography) could be used for determining the molecular weight of the glycosylated polypeptide, and then the Lowry assay and a phenol-sulfuric acid assay could be used to determine the amount of amino acid and saccharide material, respectively.

“Glycosylated” means that carbohydrate moiety is covalently bound to a molecule of interest. In embodiments, a glycosylated polypeptide means a polypeptide that has a carbohydrate moiety covalently bound to it.

“HA1 subunit of human influenza A virus hemagglutinin” means the longer (approx. 320-350 amino acids) of two disulfide-linked Human Influenza A virus hemagglutinin (HA) glycoprotein chains formed during HA maturation by proteolytic cleavage of the common HA precursor HA0, and located at the N-terminal part of HA0 (corresponding to 5′-terminal part of full HA gene, encoded by segment 4 of the influenza genome).

“HA2 subunit of human influenza A virus hemagglutinin” means the shorter (approx. 220 amino acids) of two disulfide-linked HA glycoprotein chains formed during HA maturation by proteolytic cleavage of the common HA precursor HA0, located at the C-terminal part of HA0 (corresponding to 3′-terminal part of full HA gene, encoded by segment 4 of the influenza genome).

“Human Influenza A virus hemagglutinin” or “HA” means a major envelope glycoprotein of human A influenza virus encoded by segment 4 of influenza RNA genome. Influenza HA exists in nature as a trimer composed of three identical monomers assembled into a central-helical coiled coil that consists of stem (stalk) region and three globular domains containing binding sites for surface cell receptor, essential for virus attachment to the target cell. These HA monomers are composed of two disulfide-linked chains, the HA1 subunit of human influenza A virus hemagglutinin and the HA2 subunit of human influenza A virus hemagglutinin, which are created by proteolytic cleavage of HA0 precursor during viral maturation. Aside from cell attachment, HA plays an essential role in infection by initiating a pH-dependent fusion of viral and endosomal membranes upon endocytosis. This fusion process induces dramatic conformational changes in HA2, which involve translocations of several HA2 domains, exposure of fusion peptides and formation of hydrophobic bonds between HA and the target membrane (Cross et al. 2009; Isin et al., 2002; Ekiert et al., 2009).

“Isolated nucleic acid” means a nucleic acid that is separated from its native environment and present in sufficient quantity to permit its identification or use. An isolated nucleic acid may be one that is (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is one which is readily manipulable by recombinant DNA techniques well known in the art. Thus, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. An isolated nucleic acid may be substantially purified, but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulable by standard techniques known to those of ordinary skill in the art. Any of the nucleic acids provided herein may be isolated. Any of the antigens provided herein may be provided as a nucleic acid that encodes it, and such nucleic acid may also be isolated.

“Isolated polypeptide” means the polypeptide is separated from its native environment and present in sufficient quantity to permit its identification or use. This means, for example, the polypeptide may be (i) selectively produced by expression cloning or (ii) purified as by chromatography or electrophoresis. Isolated polypeptides may be, but need not be, substantially pure. Because an isolated polypeptide may be admixed with a pharmaceutically acceptable carrier in a pharmaceutical preparation, the polypeptide may comprise only a small percentage by weight of the preparation. The polypeptide is nonetheless isolated in that it has been separated from the substances with which it may be associated in living systems, i.e., isolated from other proteins, etc. Any of the polypeptides provided herein may be isolated.

“Maximum dimension of a synthetic nanocarrier” means the largest dimension of a nanocarrier measured along any axis of the synthetic nanocarrier. “Minimum dimension of a synthetic nanocarrier” means the smallest dimension of a synthetic nanocarrier measured along any axis of the synthetic nanocarrier. For example, for a spheroidal synthetic nanocarrier, the maximum and minimum dimension of a synthetic nanocarrier would be substantially identical, and would be the size of its diameter. Similarly, for a cuboidal synthetic nanocarrier, the minimum dimension of a synthetic nanocarrier would be the smallest of its height, width or length, while the maximum dimension of a synthetic nanocarrier would be the largest of its height, width or length. In an embodiment, a minimum dimension of at least 75%, preferably at least 80%, more preferably at least 90%, of the synthetic nanocarriers in a sample, based on the total number of synthetic nanocarriers in the sample, is greater than 100 nm. In an embodiment, a maximum dimension of at least 75%, preferably at least 80%, more preferably at least 90%, of the synthetic nanocarriers in a sample, based on the total number of synthetic nanocarriers in the sample, is equal to or less than 5 μm. Preferably, a minimum dimension of at least 75%, preferably at least 80%, more preferably at least 90%, of the synthetic nanocarriers in a sample, based on the total number of synthetic nanocarriers in the sample, is greater than 110 nm, more preferably greater than 120 nm, more preferably greater than 130 nm, and more preferably still greater than 150 nm. Aspects ratios of the maximum and minimum dimensions of inventive synthetic nanocarriers may vary depending on the embodiment. For instance, aspect ratios of the maximum to minimum dimensions of the synthetic nanocarriers may vary from 1:1 to 1,000, 000:1, preferably from 1:1 to 100, 000:1, more preferably from 1:1 to 1000:1, still preferably from 1:1 to 100:1, and yet more preferably from 1:1 to 10:1. Preferably, a maximum dimension of at least 75%, preferably at least 80%, more preferably at least 90%, of the synthetic nanocarriers in a sample, based on the total number of synthetic nanocarriers in the sample is equal to or less than 3 μm, more preferably equal to or less than 2 μm, more preferably equal to or less than 1 μm, more preferably equal to or less than 800 nm, more preferably equal to or less than 600 nm, and more preferably still equal to or less than 500 nm. In preferred embodiments, a maximum dimension of at least 75%, preferably at least 80%, more preferably at least 90%, of the synthetic nanocarriers in a sample, based on the total number of synthetic nanocarriers in the sample, is equal to or greater than 100 nm, more preferably equal to or greater than 120 nm, more preferably equal to or greater than 130 nm, more preferably equal to or greater than 140 nm, and more preferably still equal to or greater than 150 nm. Measurement of synthetic nanocarrier sizes is obtained by suspending the synthetic nanocarriers in a liquid (usually aqueous) media and using dynamic light scattering (DLS) (e.g. using a Brookhaven ZetaPALS instrument). For example, a suspension of synthetic nanocarriers can be diluted from an aqueous buffer into purified water to achieve a final synthetic nanocarrier suspension concentration of approximately 0.01 to 0.1 mg/mL. The diluted suspension may be prepared directly inside, or transferred to, a suitable cuvette for DLS analysis. The cuvette may then be placed in the DLS, allowed to equilibrate to the controlled temperature, and then scanned for sufficient time to aquire a stable and reproducible distribution based on appropriate inputs for viscosity of the medium and refractive indicies of the sample. The effective diameter, or mean of the distribution, is then reported.

“Obtained” means taken from a source without substantial modification. Substantial modification is modification that significantly affects the chemical or immunological properties of the material in question. For example, as a non-limiting example, a polypeptide or nucleic acid with a sequence with greater than 90%, preferably greater than 95%, preferably greater than 97%, preferably greater than 98%, preferably greater than 99%, preferably 100%, identity to a natural polypeptide or nucleotide sequence, preferably a natural consensus polypeptide or nucleotide sequence, and chemical and/or immunological properties that are not significantly different from the natural polypeptide or nucleic acid would be said to be obtained from the natural polypeptide or nucleotide sequence. These chemical or immunological properties comprise hydrophilicity, stability, affinity, and ability to couple with a carrier such as a synthetic nanocarrier.

“Polypeptide” means a compound comprising greater than about 100 amino acids. Polypeptides according to the invention may be obtained or derived from a variety of sources, preferably from human influenza A virus hemagglutinin.

“Pharmaceutically acceptable excipient” means a pharmacologically inactive material used together with the recited synthetic nanocarriers to formulate the inventive compositions. Pharmaceutically acceptable excipients comprise a variety of materials known in the art, including but not limited to saccharides (such as glucose, lactose, and the like), preservatives such as antimicrobial agents, reconstitution aids, colorants, saline (such as phosphate buffered saline), and buffers.

“Subject” means animals, including warm blooded mammals such as humans and primates; avians; domestic household or farm animals such as cats, dogs, sheep, goats, cattle, horses and pigs; laboratory animals such as mice, rats and guinea pigs; fish; reptiles; zoo and wild animals; and the like.

“Synthetic nanocarrier(s)” means a discrete object that is not found in nature, and that possesses at least one dimension that is less than or equal to 5 microns in size. Albumin nanoparticles are generally included as synthetic nanocarriers, however in certain embodiments the synthetic nanocarriers do not comprise albumin nanoparticles. In embodiments, inventive synthetic nanocarriers do not comprise chitosan. In embodiments, synthetic nanocarriers are present in an amount sufficient to provide an immune response to the peptide upon administration of the composition to a subject. In embodiments, amounts of the synthetic nanocarriers may range from 0.1 micrograms to 500 micrograms, preferably from 1 micrograms to 100 micrograms.

A synthetic nanocarrier can be, but is not limited to, one or a plurality of lipid-based nanoparticles, polymeric nanoparticles, metallic nanoparticles, surfactant-based emulsions, dendrimers, buckyballs, nanowires, virus-like particles, peptide or protein-based particles (such as albumin nanoparticles) and/or nanoparticles that are developed using a combination of nanomaterials such as lipid-polymer nanoparticles. Synthetic nanocarriers may be a variety of different shapes, including but not limited to spheroidal, cuboidal, pyramidal, oblong, cylindrical, toroidal, and the like. Synthetic nanocarriers according to the invention comprise one or more surfaces. Exemplary synthetic nanocarriers that can be adapted for use in the practice of the present invention comprise: (1) the biodegradable nanoparticles disclosed in U.S. Pat. No. 5,543,158 to Gref et al., (2) the polymeric nanoparticles of Published US Patent Application 20060002852 to Saltzman et al., (3) the lithographically constructed nanoparticles of Published US Patent Application 20090028910 to DeSimone et al., (4) the disclosure of WO 2009/051837 to von Andrian et al., or (5) the nanoparticles disclosed in Published US Patent Application 2008/0145441 to Penades et al., (6) the protein nanoparticles disclosed in Published US Patent Application 20090226525 to de los Rios et al., (7) the virus-like particles disclosed in published US Patent Application 20060222652 to Sebbel et al., (8) the nucleic acid coupled virus-like particles disclosed in published US Patent Application 20060251677 to Bachmann et al., (9) the virus-like particles disclosed in WO2010047839A1 or WO2009106999A2, or (10) the nanoprecipitated nanoparticles disclosed in P. Paolicelli et al., “Surface-modified PLGA-based Nanoparticles that can Efficiently Associate and Deliver Virus-like Particles” Nanomedicine. 5(6):843-853 (2010). In embodiments, synthetic nanocarriers may possess an aspect ratio greater than 1:1, 1:1.2, 1:1.5, 1:2, 1:3, 1:5, 1:7, or greater than 1:10.

Synthetic nanocarriers according to the invention that have a minimum dimension of equal to or less than about 100 nm, preferably equal to or less than 100 nm, do not comprise a surface with hydroxyl groups that activate complement or alternatively comprise a surface that consists essentially of moieties that are not hydroxyl groups that activate complement. In a preferred embodiment, synthetic nanocarriers according to the invention that have a minimum dimension of equal to or less than about 100 nm, preferably equal to or less than 100 nm, do not comprise a surface that substantially activates complement or alternatively comprise a surface that consists essentially of moieties that do not substantially activate complement. In a more preferred embodiment, synthetic nanocarriers according to the invention that have a minimum dimension of equal to or less than about 100 nm, preferably equal to or less than 100 nm, do not comprise a surface that activates complement or alternatively comprise a surface that consists essentially of moieties that do not activate complement. In embodiments, synthetic nanocarriers exclude virus-like particles. In embodiments, when synthetic nanocarriers comprise virus-like particles, the virus-like particles comprise non-natural adjuvant (meaning that the VLPs comprise an adjuvant other than naturally occurring RNA generated during the production of the VLPs). In embodiments, synthetic nanocarriers may possess an aspect ratio greater than 1:1, 1:1.2, 1:1.5, 1:2, 1:3, 1:5, 1:7, or greater than 1:10.

“T cell antigen” means any antigen that is recognized by and triggers an immune response in a T cell (e.g., an antigen that is specifically recognized by a T cell receptor on a T cell or an NKT cell via presentation of the antigen or portion thereof bound to a Class I or Class II major histocompatability complex molecule (MHC), or bound to a CD1 complex. In some embodiments, an antigen that is a T cell antigen is also a B cell antigen. In other embodiments, the T cell antigen is not also a B cell antigen. T cell antigens generally are proteins, polypeptides or peptides. T cell antigens may be an antigen that stimulates a CD8+ T cell response, a CD4+ T cell response, or both. The nanocarriers, therefore, in some embodiments can effectively stimulate both types of responses.

In some embodiments the T cell antigen is a T helper cell antigen (i.e. one that can generate an enhanced response to a B cell antigen, preferably an unrelated B cell antigen, through stimulation of T cell help). In embodiments, a T helper cell antigen may comprise one or more peptides obtained or derived from tetanus toxoid, Epstein-Barr virus, influenza virus, respiratory syncytial virus, measles virus, mumps virus, rubella virus, cytomegalovirus, adenovirus, diphtheria toxoid, or a PADRE peptide (known from the work of Sette et al. U.S. Pat. No. 7,202,351). In other embodiments, a T helper cell antigen may comprise one or more lipids, or glycolipids, including but not limited to: α-galactosylceramide (α-GalCer), α-linked glycosphingolipids (from Sphingomonas spp.), galactosyl diacylglycerols (from Borrelia burgdorferi), lypophosphoglycan (from Leishmania donovani), and phosphatidylinositol tetramannoside (PIM4) (from Mycobacterium leprae). For additional lipids and/or glycolipids useful as a T helper cell antigen, see V. Cerundolo et al., “Harnessing invariant NKT cells in vaccination strategies.” Nature Rev Immun, 9:28-38 (2009). In embodiments, CD4+ T-cell antigens may be derivatives of a CD4+ T-cell antigen that is obtained from a source, such as a natural source. In such embodiments, CD4+ T-cell antigen sequences, such as those peptides that bind to MHC II, may have at least 70%, 80%, 90%, or 95% identity to the antigen obtained from the source. In embodiments, the T cell antigen, preferably a T helper cell antigen, may be coupled to, or uncoupled from, a synthetic nanocarrier.

“Vaccine” means a composition of matter that improves the immune response to a particular pathogen or disease. A vaccine typically contains factors that stimulate a subject's immune system to recognize a specific antigen as foreign and eliminate it from the subject's body. A vaccine also establishes an immunologic ‘memory’ so the antigen will be quickly recognized and responded to if a person is re-challenged. Vaccines can be prophylactic (for example to prevent future infection by any pathogen), or therapeutic (for example a vaccine against a tumor specific antigen for the treatment of cancer). In embodiments, a vaccine may comprise dosage forms according to the invention.

### C. Inventive Compositions

Human Influenza A virus hemagglutinin, HA1 subunit of human influenza A virus hemagglutinin, or HA2 subunit of human influenza A virus hemagglutinin may be obtained using conventional means. In one embodiment, these materials may be produced recombinantly, using a mammalian or insect protein production system. In a preferred embodiment, recombinant Full-Length H5N1 A/Vietnam/1203/04 is glycosylated with N-linked sugars, produced using the baculovirus expression vector system with a molecular weight of about 72,000, and is available from Protein Sciences Corporation (Meriden Conn.). The HA1 and/or HA2 subunits may be obtained from the HA material, such as the Protein Sciences prep noted above. The molecular weight of the HA1 and HA2 subunit material, obtained using the Protein Sciences prep, is noted to be approximately 58,000 and 28,000 MW, respectively. The HA, HAL or HA2 can be readied for conjugation to the inventive synthetic nanocarriers using the coupling methods discussed elsewhere herein, and then coupled to the synthetic nanocarriers using those coupling methods. In some embodiments, at least a portion of the polypeptides obtained or derived from human influenza A virus hemagglutinin are coupled to a surface of the synthetic nanocarriers. In additional embodiments, the synthetic nanocarriers are covalently, or are non-covalently, coupled to polypeptides obtained or derived from human influenza A virus hemagglutinin.

A wide variety of synthetic nanocarriers can be used according to the invention. In some embodiments, synthetic nanocarriers are spheres or spheroids. In some embodiments, synthetic nanocarriers are flat or plate-shaped. In some embodiments, synthetic nanocarriers are cubes or cubic. In some embodiments, synthetic nanocarriers are ovals or ellipses. In some embodiments, synthetic nanocarriers are cylinders, cones, or pyramids.

In some embodiments, it is desirable to use a population of synthetic nanocarriers that is relatively uniform in terms of size, shape, and/or composition so that each synthetic nanocarrier has similar properties. For example, at least 80%, at least 90%, or at least 95% of the synthetic nanocarriers, based on the total number of synthetic nanocarriers, may have a minimum dimension or maximum dimension that falls within 5%, 10%, or 20% of the average diameter or average dimension of the synthetic nanocarriers. In some embodiments, a population of synthetic nanocarriers may be heterogeneous with respect to size, shape, and/or composition.

Synthetic nanocarriers can be solid or hollow and can comprise one or more layers. In some embodiments, each layer has a unique composition and unique properties relative to the other layer(s). To give but one example, synthetic nanocarriers may have a core/shell structure, wherein the core is one layer (e.g. a polymeric core) and the shell is a second layer (e.g. a lipid bilayer or monolayer). Synthetic nanocarriers may comprise a plurality of different layers.

In some embodiments, synthetic nanocarriers may optionally comprise one or more lipids. In some embodiments, a synthetic nanocarrier may comprise a liposome. In some embodiments, a synthetic nanocarrier may comprise a lipid bilayer. In some embodiments, a synthetic nanocarrier may comprise a lipid monolayer. In some embodiments, a synthetic nanocarrier may comprise a micelle. In some embodiments, a synthetic nanocarrier may comprise a core comprising a polymeric matrix surrounded by a lipid layer (e.g., lipid bilayer, lipid monolayer, etc.). In some embodiments, a synthetic nanocarrier may comprise a non-polymeric core (e.g., metal particle, quantum dot, ceramic particle, bone particle, viral particle, proteins, nucleic acids, carbohydrates, etc.) surrounded by a lipid layer (e.g., lipid bilayer, lipid monolayer, etc.).

In some embodiments, synthetic nanocarriers can comprise one or more polymers. In some embodiments, such a polymer can be surrounded by a coating layer (e.g., liposome, lipid monolayer, micelle, etc.). In some embodiments, various elements of the synthetic nanocarriers can be coupled with the polymer.

In some embodiments, an immunofeature surface, targeting moiety, and/or oligonucleotide (or other element) can be covalently associated with a polymeric matrix. In some embodiments, covalent association is mediated by a linker. In some embodiments, an immunofeature surface, targeting moiety, and/or oligonucleotide (or other element) can be noncovalently associated with a polymeric matrix. For example, in some embodiments, an immunofeature surface, targeting moiety, and/or oligonucleotide (or other element) can be encapsulated within, surrounded by, and/or dispersed throughout a polymeric matrix. Alternatively or additionally, an immunofeature surface, targeting moiety, and/or nucleotide (or other element) can be associated with a polymeric matrix by hydrophobic interactions, charge interactions, van der Waals forces, etc.

A wide variety of polymers and methods for forming polymeric matrices therefrom are known conventionally. In general, a polymeric matrix comprises one or more polymers. Polymers may be natural or unnatural (synthetic) polymers. Polymers may be homopolymers or copolymers comprising two or more monomers. In terms of sequence, copolymers may be random, block, or comprise a combination of random and block sequences. Typically, polymers in accordance with the present invention are organic polymers.

Examples of polymers suitable for use in the present invention include, but are not limited to polyethylenes, polycarbonates (e.g. poly(1,3-dioxan-2one)), polyanhydrides (e.g. poly(sebacic anhydride)), polypropylfumerates, polyamides (e.g. polycaprolactam), polyacetals, polyethers, polyesters (e.g., polylactide, polyglycolide, polylactide-co-glycolide, polycaprolactone, polyhydroxyacid (e.g. poly(β-hydroxyalkanoate))), poly(orthoesters), polycyanoacrylates, polyvinyl alcohols, polyurethanes, polyphosphazenes, polyacrylates, polymethacrylates, polyureas, polystyrenes, and polyamines, polylysine, polylysine-PEG copolymers, and poly(ethyleneimine), poly(ethylene imine)-PEG copolymers.

In some embodiments, polymers in accordance with the present invention include polymers which have been approved for use in humans by the U.S. Food and Drug Administration (FDA) under 21 C.F.R. §177.2600, including but not limited to polyesters (e.g., polylactic acid, poly(lactic-co-glycolic acid), polycaprolactone, polyvalerolactone, poly(1,3-dioxan-2one)); polyanhydrides (e.g., poly(sebacic anhydride)); polyethers (e.g., polyethylene glycol); polyurethanes; polymethacrylates; polyacrylates; and polycyanoacrylates.

In some embodiments, polymers can be hydrophilic. For example, polymers may comprise anionic groups (e.g., phosphate group, sulphate group, carboxylate group); cationic groups (e.g., quaternary amine group); or polar groups (e.g., hydroxyl group, thiol group, amine group). In some embodiments, a synthetic nanocarrier comprising a hydrophilic polymeric matrix generates a hydrophilic environment within the synthetic nanocarrier. In some embodiments, polymers can be hydrophobic. In some embodiments, a synthetic nanocarrier comprising a hydrophobic polymeric matrix generates a hydrophobic environment within the synthetic nanocarrier. Selection of the hydrophilicity or hydrophobicity of the polymer may have an impact on the nature of materials that are incorporated (e.g. coupled) within the synthetic nanocarrier.

In some embodiments, polymers may be modified with one or more moieties and/or functional groups. A variety of moieties or functional groups can be used in accordance with the present invention. In some embodiments, polymers may be modified with polyethylene glycol (PEG), with a carbohydrate, and/or with acyclic polyacetals derived from polysaccharides (Papisov, 2001, ACS Symposium Series, 786:301). Certain embodiments may be made using the general teachings of U.S. Pat. No. 5,543,158 to Gref et al., or WO publication WO2009/051837 by Von Andrian et al.

In some embodiments, polymers may be modified with a lipid or fatty acid group. In some embodiments, a fatty acid group may be one or more of butyric, caproic, caprylic, capric, lauric, myristic, palmitic, stearic, arachidic, behenic, or lignoceric acid. In some embodiments, a fatty acid group may be one or more of palmitoleic, oleic, vaccenic, linoleic, alpha-linoleic, gamma-linoleic, arachidonic, gadoleic, arachidonic, eicosapentaenoic, docosahexaenoic, or erucic acid.

In some embodiments, polymers may be polyesters, including copolymers comprising lactic acid and glycolic acid units, such as poly(lactic acid-co-glycolic acid) and poly(lactide-co-glycolide), collectively referred to herein as “PLGA”; and homopolymers comprising glycolic acid units, referred to herein as “PGA,” and lactic acid units, such as poly-L-lactic acid, poly-D-lactic acid, poly-D,L-lactic acid, poly-L-lactide, poly-D-lactide, and poly-D,L-lactide, collectively referred to herein as “PLA.” In some embodiments, exemplary polyesters include, for example, polyhydroxyacids; PEG copolymers and copolymers of lactide and glycolide (e.g., PLA-PEG copolymers, PGA-PEG copolymers, PLGA-PEG copolymers, and derivatives thereof. In some embodiments, polyesters include, for example, poly(caprolactone), poly(caprolactone)-PEG copolymers, poly(L-lactide-co-L-lysine), poly(serine ester), poly(4-hydroxy-L-proline ester), poly[α-(4-aminobutyl)-L-glycolic acid], and derivatives thereof.

In some embodiments, a polymer may be PLGA. PLGA is a biocompatible and biodegradable co-polymer of lactic acid and glycolic acid, and various forms of PLGA are characterized by the ratio of lactic acid:glycolic acid. Lactic acid can be L-lactic acid, D-lactic acid, or D,L-lactic acid. The degradation rate of PLGA can be adjusted by altering the lactic acid:glycolic acid ratio. In some embodiments, PLGA to be used in accordance with the present invention is characterized by a lactic acid:glycolic acid ratio of approximately 85:15, approximately 75:25, approximately 60:40, approximately 50:50, approximately 40:60, approximately 25:75, or approximately 15:85.

In some embodiments, polymers may be one or more acrylic polymers. In certain embodiments, acrylic polymers include, for example, acrylic acid and methacrylic acid copolymers, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamide copolymer, poly(methyl methacrylate), poly(methacrylic acid anhydride), methyl methacrylate, polymethacrylate, poly(methyl methacrylate) copolymer, polyacrylamide, aminoalkyl methacrylate copolymer, glycidyl methacrylate copolymers, polycyanoacrylates, and combinations comprising one or more of the foregoing polymers. The acrylic polymer may comprise fully-polymerized copolymers of acrylic and methacrylic acid esters with a low content of quaternary ammonium groups.

In some embodiments, polymers can be cationic polymers. In general, cationic polymers are able to condense and/or protect negatively charged strands of nucleic acids (e.g. DNA, or derivatives thereof). Amine-containing polymers such as poly(lysine) (Zauner et al., 1998, Adv. Drug Del. Rev., 30:97; and Kabanov et al., 1995, Bioconjugate Chem., 6:7), poly(ethylene imine) (PEI; Boussif et al., 1995, Proc. Natl. Acad. Sci., USA, 1995, 92:7297), and poly(amidoamine) dendrimers (Kukowska-Latallo et al., 1996, Proc. Natl. Acad. Sci., USA, 93:4897; Tang et al., 1996, Bioconjugate Chem., 7:703; and Haensler et al., 1993, Bioconjugate Chem., 4:372) are positively-charged at physiological pH, form ion pairs with nucleic acids, and mediate transfection in a variety of cell lines. In embodiments, the inventive synthetic nanocarriers may not comprise (or may exclude) cationic polymers.

In some embodiments, polymers can be degradable polyesters bearing cationic side chains (Putnam et al., 1999, Macromolecules, 32:3658; Barrera et al., 1993, J. Am. Chem. Soc., 115:11010; Kwon et al., 1989, Macromolecules, 22:3250; Lim et al., 1999, J. Am. Chem. Soc., 121:5633; and Zhou et al., 1990, Macromolecules, 23:3399). Examples of these polyesters include poly(L-lactide-co-L-lysine) (Barrera et al., 1993, J. Am. Chem. Soc., 115:11010), poly(serine ester) (Zhou et al., 1990, Macromolecules, 23:3399), poly(4-hydroxy-L-proline ester) (Putnam et al., 1999, Macromolecules, 32:3658; and Lim et al., 1999, J. Am. Chem. Soc., 121:5633), and poly(4-hydroxy-L-proline ester) (Putnam et al., 1999, Macromolecules, 32:3658; and Lim et al., 1999, J. Am. Chem. Soc., 121:5633).

The properties of these and other polymers and methods for preparing them are well known in the art (see, for example, U.S. Pat. Nos. 6,123,727; 5,804,178; 5,770,417; 5,736,372; 5,716,404; 6,095,148; 5,837,752; 5,902,599; 5,696,175; 5,514,378; 5,512,600; 5,399,665; 5,019,379; 5,010,167; 4,806,621; 4,638,045; and 4,946,929; Wang et al., 2001, J. Am. Chem. Soc., 123:9480; Lim et al., 2001, J. Am. Chem. Soc., 123:2460; Langer, 2000, Acc. Chem. Res., 33:94; Langer, 1999, J. Control. Release, 62:7; and Uhrich et al., 1999, Chem. Rev., 99:3181). More generally, a variety of methods for synthesizing certain suitable polymers are described in Concise Encyclopedia of Polymer Science and Polymeric Amines and Ammonium Salts, Ed. by Goethals, Pergamon Press, 1980; Principles of Polymerization by Odian, John Wiley & Sons, Fourth Edition, 2004; Contemporary Polymer Chemistry by Allcock et al., Prentice-Hall, 1981; Deming et al., 1997, Nature, 390:386; and in U.S. Pat. Nos. 6,506,577, 6,632,922, 6,686,446, and 6,818,732.

In some embodiments, polymers can be linear or branched polymers. In some embodiments, polymers can be dendrimers. In some embodiments, polymers can be substantially cross-linked to one another. In some embodiments, polymers can be substantially free of cross-links. In some embodiments, polymers can be used in accordance with the present invention without undergoing a cross-linking step. It is further to be understood that inventive synthetic nanocarriers may comprise block copolymers, graft copolymers, blends, mixtures, and/or adducts of any of the foregoing and other polymers. Those skilled in the art will recognize that the polymers listed herein represent an exemplary, not comprehensive, list of polymers that can be of use in accordance with the present invention.

In some embodiments, synthetic nanocarriers do not comprise a polymeric component. In some embodiments, synthetic nanocarriers may comprise metal particles, quantum dots, ceramic particles, etc. In some embodiments, a non-polymeric synthetic nanocarrier is an aggregate of non-polymeric components, such as an aggregate of metal atoms (e.g., gold atoms).

In some embodiments, synthetic nanocarriers may optionally comprise one or more amphiphilic entities. In some embodiments, an amphiphilic entity can promote the production of synthetic nanocarriers with increased stability, improved uniformity, or increased viscosity. In some embodiments, amphiphilic entities can be associated with the interior surface of a lipid membrane (e.g., lipid bilayer, lipid monolayer, etc.). Many amphiphilic entities known in the art are suitable for use in making synthetic nanocarriers in accordance with the present invention. Such amphiphilic entities include, but are not limited to, phosphoglycerides; phosphatidylcholines; dipalmitoyl phosphatidylcholine (DPPC); dioleylphosphatidyl ethanolamine (DOPE); dioleyloxypropyltriethylammonium (DOTMA); dioleoylphosphatidylcholine; cholesterol; cholesterol ester; diacylglycerol; diacylglycerolsuccinate; diphosphatidyl glycerol (DPPG); hexanedecanol; fatty alcohols such as polyethylene glycol (PEG); polyoxyethylene-9-lauryl ether; a surface active fatty acid, such as palmitic acid or oleic acid; fatty acids; fatty acid monoglycerides; fatty acid diglycerides; fatty acid amides; sorbitan trioleate (Span®85) glycocholate; sorbitan monolaurate (Span®20); polysorbate 20 (Tween®20); polysorbate 60 (Tween®60); polysorbate 65 (Tween®65); polysorbate 80 (Tween®80); polysorbate 85 (Tween®85); polyoxyethylene monostearate; surfactin; a poloxomer; a sorbitan fatty acid ester such as sorbitan trioleate; lecithin; lysolecithin; phosphatidylserine; phosphatidylinositol; sphingomyelin; phosphatidylethanolamine (cephalin); cardiolipin; phosphatidic acid; cerebrosides; dicetylphosphate; dipalmitoylphosphatidylglycerol; stearylamine; dodecylamine; hexadecyl-amine; acetyl palmitate; glycerol ricinoleate; hexadecyl sterate; isopropyl myristate; tyloxapol; poly(ethylene glycol)5000-phosphatidylethanolamine; poly(ethylene glycol)400-monostearate; phospholipids; synthetic and/or natural detergents having high surfactant properties; deoxycholates; cyclodextrins; chaotropic salts; ion pairing agents; and combinations thereof. An amphiphilic entity component may be a mixture of different amphiphilic entities. Those skilled in the art will recognize that this is an exemplary, not comprehensive, list of substances with surfactant activity. Any amphiphilic entity may be used in the production of synthetic nanocarriers to be used in accordance with the present invention.

In some embodiments, synthetic nanocarriers may optionally comprise one or more carbohydrates. Carbohydrates may be natural or synthetic. A carbohydrate may be a derivatized natural carbohydrate. In certain embodiments, a carbohydrate comprises monosaccharide or disaccharide, including but not limited to glucose, fructose, galactose, ribose, lactose, sucrose, maltose, trehalose, cellbiose, mannose, xylose, arabinose, glucoronic acid, galactoronic acid, mannuronic acid, glucosamine, galatosamine, and neuramic acid. In certain embodiments, a carbohydrate is a polysaccharide, including but not limited to pullulan, cellulose, microcrystalline cellulose, hydroxypropyl methylcellulose (HPMC), hydroxycellulose (HC), methylcellulose (MC), dextran, cyclodextran, glycogen, hydroxyethylstarch, carageenan, glycon, amylose, chitosan, N,O-carboxylmethylchitosan, algin and alginic acid, starch, chitin, inulin, konjac, glucommannan, pustulan, heparin, hyaluronic acid, curdlan, and xanthan. In embodiments, the inventive synthetic nanocarriers do not comprise (or specifically exclude) carbohydrates, such as a polysaccharide. In certain embodiments, the carbohydrate may comprise a carbohydrate derivative such as a sugar alcohol, including but not limited to mannitol, sorbitol, xylitol, erythritol, maltitol, and lactitol.

Compositions according to the invention comprise inventive synthetic nanocarriers in combination with pharmaceutically acceptable excipients, such as preservatives, buffers, saline, or phosphate buffered saline. The compositions may be made using conventional pharmaceutical manufacturing and compounding techniques to arrive at useful dosage forms. Inventive compositions may comprise inorganic or organic buffers (e.g., sodium or potassium salts of phosphate, carbonate, acetate, or citrate) and pH adjustment agents (e.g., hydrochloric acid, sodium or potassium hydroxide, salts of citrate or acetate, amino acids and their salts) antioxidants (e.g., ascorbic acid, alpha-tocopherol), surfactants (e.g., polysorbate 20, polysorbate 80, polyoxyethylene9-10 nonyl phenol, sodium desoxycholate), solution and/or cryo/lyo stabilizers (e.g., sucrose, lactose, mannitol, trehalose), osmotic adjustment agents (e.g., salts or sugars), antibacterial agents (e.g., benzoic acid, phenol, gentamicin), antifoaming agents (e.g., polydimethylsilozone), preservatives (e.g., thimerosal, 2-phenoxyethanol, EDTA), polymeric stabilizers and viscosity-adjustment agents (e.g., polyvinylpyrrolidone, poloxamer 488, carboxymethylcellulose) and co-solvents (e.g., glycerol, polyethylene glycol, ethanol). In an embodiment, inventive synthetic nanocarriers are suspended in sterile saline solution for injection together with a preservative.

In embodiments, when preparing synthetic nanocarriers as carriers for adjuvants for use in vaccines, methods for coupling the adjuvants to the synthetic nanocarriers may be useful. If the adjuvant is a small molecule it may be of advantage to attach the adjuvant to a polymer prior to the assembly of the synthetic nanocarriers. In embodiments, it may also be an advantage to prepare the synthetic nanocarriers with surface groups that are used to couple the adjuvant to the synthetic nanocarrier through the use of these surface groups rather than attaching the adjuvant to a polymer and then using this polymer conjugate in the construction of synthetic nanocarriers.

The recited polypeptides can be coupled to the synthetic nanocarriers by a variety of methods. In embodiments, the recited polypeptide is coupled to an external surface of the synthetic nanocarrier covalently or non-covalently.

In certain embodiments, the coupling can be a covalent linker. In embodiments, polypeptides according to the invention can be covalently coupled to the external surface via a 1,2,3-triazole linker formed by the 1,3-dipolar cycloaddition reaction of azido groups on the surface of the nanocarrier with polypeptides containing an alkyne group or by the 1,3-dipolar cycloaddition reaction of alkynes on the surface of the nanocarrier with polypeptides containing an azido group. Such cycloaddition reactions are preferably performed in the presence of a Cu(I) catalyst along with a suitable Cu(I)-ligand and a reducing agent to reduce Cu(II) compound to catalytic active Cu(I) compound. This Cu(I)-catalyzed azide-alkyne cycloaddition (CuAAC) can also be referred as the click reaction.

Additionally, the covalent coupling may comprise a covalent linker that comprises an amide linker, a disulfide linker, a thioether linker, a hydrazone linker, a hydrazide linker, an imine or oxime linker, an urea or thiourea linker, an amidine linker, an amine linker, and a sulfonamide linker.

An amide linker is formed via an amide bond between an amine on one component such as the polypeptide with the carboxylic acid group of a second component such as the nanocarrier. The amide bond in the linker can be made using any of the conventional amide bond forming reactions with suitably protected amino acids or polypeptides and activated carboxylic acid such N-hydroxysuccinimide-activated ester.

A disulfide linker is made via the formation of a disulfide (S—S) bond between two sulfur atoms of the form, for instance, of R₁—S—S—R₂. A disulfide bond can be formed by thiol exchange of a polypeptide containing thiol/mercaptan group (—SH) with another activated thiol group on a polymer or nanocarrier or a nanocarrier containing thiol/mercaptan groups with a polypeptide containing activated thiol group.

A triazole linker, specifically a 1,2,3-triazole of the form

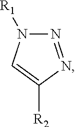

wherein R₁ and R₂ may be any chemical entities, is made by the 1,3-dipolar cycloaddition reaction of an azide attached to a first component such as the nanocarrier with a terminal alkyne attached to a second component such as the polypeptide. The 1,3-dipolar cycloaddition reaction is performed with or without a catalyst, preferably with Cu(I)-catalyst, which links the two components through a 1,2,3-triazole function. This chemistry is described in detail by Sharpless et al., Angew. Chem. Int. Ed. 41(14), 2596, (2002) and Meldal, et al, Chem. Rev., 2008, 108(8), 2952-3015 and is often referred to as a “click” reaction or CuAAC.

In embodiments, a polymer containing an azide or alkyne group, terminal to the polymer chain is prepared. This polymer is then used to prepare a synthetic nanocarrier in such a manner that a plurality of the alkyne or azide groups are positioned on the surface of that nanocarrier. Alternatively, the synthetic nanocarrier can be prepared by another route, and subsequently functionalized with alkyne or azide groups. The polypeptide is prepared with the presence of either an alkyne (if the polymer contains an azide) or an azide (if the polymer contains an alkyne) group. The polypeptide is then allowed to react with the nanocarrier via the 1,3-dipolar cycloaddition reaction with or without a catalyst which covalently couples the polypetide to the particle through the 1,4-disubstituted 1,2,3-triazole linker.

A thioether linker is made by the formation of a sulfur-carbon (thioether) bond in the form, for instance, of R₁—S—R₂. Thioether can be made by either alkylation of a thiol/mercaptan (—SH) group on one component such as the polypeptide with an alkylating group such as halide or epoxide on a second component such as the nanocarrier. Thioether linkers can also be formed by Michael addition of a thiol/mercaptan group on one component such as a polypeptide to an electron-deficient alkene group on a second component such as a polymer containing a maleimide group or vinyl sulfone group as the Michael acceptor. In another way, thioether linkers can be prepared by the radical thiol-ene reaction of a thiol/mercaptan group on one component such as a polypeptide with an alkene group on a second component such as a polymer or nanocarrier.

A hydrazone linker is made by the reaction of a hydrazide group on one component such as the polypeptide with an aldehyde/ketone group on the second component such as the nanocarrier.

A hydrazide linker is formed by the reaction of a hydrazine group on one component such as the polypeptide with a carboxylic acid group on the second component such as the nanocarrier. Such reaction is generally performed using chemistry similar to the formation of amide bond where the carboxylic acid is activated with an activating reagent.

An imine or oxime linker is formed by the reaction of an amine or N-alkoxyamine (or aminooxy) group on one component such as the polypeptide with an aldehyde or ketone group on the second component such as the nanocarrier.

An urea or thiourea linker is prepared by the reaction of an amine group on one component such as the polypeptide with an isocyanate or thioisocyanate group on the second component such as the nanocarrier.

An amidine linker is prepared by the reaction of an amine group on one component such as the polypeptide with an imidoester group on the second component such as the nanocarrier.

An amine linker is made by the alkylation reaction of an amine group on one component such as the polypeptide with an alkylating group such as halide, epoxide, or sulfonate ester group on the second component such as the nanocarrier. Alternatively, an amine linker can also be made by reductive amination of an amine group on one component such as the polypeptide with an aldehyde or ketone group on the second component such as the nanocarrier with a suitable reducing reagent such as sodium cyanoborohydride or sodium triacetoxyborohydride.

A sulfonamide linker is made by the reaction of an amine group on one component such as the polypeptide with a sulfonyl halide (such as sulfonyl chloride) group on the second component such as the nanocarrier.

A sulfone linker is made by Michael addition of a nucleophile to a vinyl sulfone. Either the vinyl sulfone or the nucleophile may be on the surface of the nanocarrier or attached to the antigen.

Additional descriptions of available conjugation methods are available in “Bioconjugate Techniques”, 2nd Edition yy Greg T. Hermanson, Published by Academic Press, Inc., 2008) (Hermanson 2008.)

The polypeptide can also be conjugated to the nanocarrier via non-covalent conjugation methods. For examples, a negative charged polypeptide can be conjugated to a positive charged nanocarrier through electrostatic adsorption. A polypeptide containing a metal ligand can also be conjugated to a nanocarrier containing a metal complex via a metal-ligand complex.

In embodiments, a polypeptide can be attached to a polymer, for example polylactic acid-block-polyethylene glycol, prior to the assembly of the synthetic nanocarrier or the synthetic nanocarrier can be formed with reactive or activatible groups on its surface. In the latter case, the polypeptide is prepared with a group that is compatible with the attachment chemistry that is presented by the synthetic nanocarriers' surface. In other embodiments, a polypeptide antigen can be attached to VLPs or liposomes using a suitable linker. A linker is a compound or reagent that capable of coupling two molecules together. In an embodiment, the linker can be a homobifuntional or heterobifunctional reagent as described in Hermanson 2008. For example, an VLP or liposome synthetic nanocarrier containing a carboxylic group on the surface can be treated with a homobifunctional linker, adipic dihydrazide (ADH), in the presence of EDC to form the corresponding synthetic nanocarrier with the ADH linker. The resulting ADH linked synthetic nanocarrier is then conjugated with a polypeptide containing an acid group via the other end of the ADH linker on NC to produce the corresponding VLP or liposome polypeptide conjugate.

In the present embodiments, a polypeptide obtained or derived from HA protein according to the invention that comprises a C-terminal alkyne group may be conjugated via the Cu(I)-catalyzed 1,3-dipolar cycloaddition (CuAAC) to synthetic nanocarriers made of PLA-PEG-azide polymer while the azide groups are on the surface of the synthetic nanocarriers. In a specific embodiment, the Cu(I) catalyst is formed in situ from CuSO4 and sodium ascorbate. Preferably, a suitable Cu(I) ligand such as Tris(3-hydroxypropyltriazolylmethyl)amine, is used to maintain the activity of the Cu(I) catalyst. The reaction is performed in buffered aq solution (pH 6-9) at 4 to 25 C over 2-48 h.

In addition to covalent attachment the polypeptide can be adsorbed to a pre-formed synthetic nanocarrier or it can be encapsulated during the formation of the synthetic nanocarrier.

In embodiments, the inventive synthetic nanocarriers may be coupled to one or more adjuvants, and/or may be coupled to a T-helper antigen. Types of adjuvants and T-helper antigens useful in the practice of the present invention have been described elsewhere. The amounts of such adjuvants and/or T-helper antigens to be included in the inventive synthetic nanocarriers may be determined using conventional dose ranging techniques. Adjuvants and/or T-helper antigens may be coupled to the synthetic nanocarriers using coupling methods disclosed elsewhere herein, or known conventionally, and adapted for use with the particular adjuvant and/or T-helper antigen (e.g. use of linker chemistries noted for use with the recited polypeptides, including the techniques of Hermanson 2008, or non-covalent coupling techniques (encapsulation, adsorption, and the like), etc., in each case adapted to the adjuvant and/or T-helper antigen of interest may also be used). Use of adjuvants and/or T-helper antigens can provide an improved immune response to the recited polypeptides.

### D. Methods of Making and Using the Inventive Compositions and Related Methods

Synthetic nanocarriers may be prepared using a wide variety of methods known in the art. For example, synthetic nanocarriers can be formed by methods as nanoprecipitation, flow focusing fluidic channels, spray drying, single and double emulsion solvent evaporation, solvent extraction, phase separation, milling, microemulsion procedures, microfabrication, nanofabrication, sacrificial layers, simple and complex coacervation, and other methods well known to those of ordinary skill in the art. Alternatively or additionally, aqueous and organic solvent syntheses for monodisperse semiconductor, conductive, magnetic, organic, and other nanomaterials have been described (Pellegrino et al., 2005, Small, 1:48; Murray et al., 2000, Ann. Rev. Mat. Sci., 30:545; and Trindade et al., 2001, Chem. Mat., 13:3843). Additional methods have been described in the literature (see, e.g., Doubrow, Ed., “Microcapsules and Nanoparticles in Medicine and Pharmacy,” CRC Press, Boca Raton, 1992; Mathiowitz et al., 1987, J. Control. Release, 5:13; Mathiowitz et al., 1987, Reactive Polymers, 6: 275; and Mathiowitz et al., 1988, J. Appl. Polymer Sci., 35:755, and also U.S. Pat. Nos. 5,578,325 and 6,007,845); P. Paolicelli et al., “Surface-modified PLGA-based Nanoparticles that can Efficiently Associate and Deliver Virus-like Particles” Nanomedicine. 5(6):843-853 (2010)).

Various materials may be coupled through encapsulation into synthetic nanocarriers as desirable using a variety of methods including but not limited to C. Astete et al., “Synthesis and characterization of PLGA nanoparticles” J. Biomater. Sci. Polymer Edn, Vol. 17, No. 3, pp. 247-289 (2006); K. Avgoustakis “Pegylated Poly(Lactide) and Poly(Lactide-Co-Glycolide) Nanoparticles: Preparation, Properties and Possible Applications in Drug Delivery” Current Drug Delivery 1:321-333 (2004); C. Reis et al., “Nanoencapsulation I. Methods for preparation of drug-loaded polymeric nanoparticles” Nanomedicine 2:8-21 (2006); P. Paolicelli et al., “Surface-modified PLGA-based Nanoparticles that can Efficiently Associate and Deliver Virus-like Particles” Nanomedicine. 5(6):843-853 (2010)). Other methods suitable for encapsulating materials, such as oligonucleotides, into synthetic nanocarriers may be used, including without limitation methods disclosed in U.S. Pat. No. 6,632,671 to Unger (Oct. 14, 2003).

In certain embodiments, synthetic nanocarriers are prepared by a nanoprecipitation process or spray drying. Conditions used in preparing synthetic nanocarriers may be altered to yield particles of a desired size or property (e.g., hydrophobicity, hydrophilicity, external morphology, “stickiness,” shape, etc.). The method of preparing the synthetic nanocarriers and the conditions (e.g., solvent, temperature, concentration, air flow rate, etc.) used may depend on the materials to be coupled to the synthetic nanocarriers and/or the composition of the polymer matrix.

If particles prepared by any of the above methods have a size range outside of the desired range, particles can be sized, for example, using a sieve.

Elements of the inventive synthetic nanocarriers—such as moieties of which an immunofeature surface is comprised, targeting moieties, polymeric matrices, antigens and the like—may be coupled to the overall synthetic nanocarrier, e.g., by one or more covalent bonds, or may be coupled by means of one or more linkers. Additional methods of functionalizing synthetic nanocarriers may be adapted from Published US Patent Application 2006/0002852 to Saltzman et al., Published US Patent Application 2009/0028910 to DeSimone et al., or Published International Patent Application WO/2008/127532 A1 to Murthy et al.

Alternatively or additionally, synthetic nanocarriers can be coupled to immunofeature surfaces, targeting moieties, adjuvants, various antigens, and/or other elements directly or indirectly via non-covalent interactions. In non-covalent embodiments, the non-covalent coupling is mediated by non-covalent interactions including but not limited to charge interactions, affinity interactions, metal coordination, physical adsorption, host-guest interactions, hydrophobic interactions, TT stacking interactions, hydrogen bonding interactions, van der Waals interactions, magnetic interactions, electrostatic interactions, dipole-dipole interactions, and/or combinations thereof. Such couplings may be arranged to be on an external surface or an internal surface of an inventive synthetic nanocarrier. In embodiments, encapsulation and/or absorbtion are forms of coupling.

Doses of dosage forms contain varying amounts of synthetic nanocarriers and varying amounts of antigens, according to the invention. The amount of synthetic nanocarriers and/or antigens present in the inventive dosage forms can be varied according to the nature of the antigens, the therapeutic benefit to be accomplished, and other such parameters. In embodiments, dose ranging studies can be conducted to establish optimal therapeutic amount of the synthetic nanocarriers and the amount of HA antigens to be present in the dosage form. In embodiments, the synthetic nanocarriers and the HA antigens are present in the dosage form in an amount effective to generate an immune response to the HA antigens upon administration to a subject. It is possible to determine amounts of the HA, or related antigens such as HA1 or HA2 (including entire or fragments of HA, HAL or HA2), effective to generate an immune response using conventional dose ranging studies and techniques in subjects. Inventive dosage forms may be administered at a variety of frequencies. In an embodiment, at least one administration of the dosage form is sufficient to generate a pharmacologically relevant response. In additional embodiments, at least two administrations, at least three administrations, or at least four administrations, of the dosage form are utilized to ensure a pharmacologically relevant response.

In embodiments, the inventive synthetic nanocarriers can be combined with other adjuvants by admixing in the same vehicle or delivery system. Such adjuvants may include, but are not limited to mineral salts, such as alum, alum combined with monphosphoryl lipid (MPL) A of Enterobacteria, such as Escherichia coli, Salmonella minnesota, Salmonella typhimurium, or Shigella flexneri or specifically with MPL® (AS04), MPL A of above-mentioned bacteria separately, saponins, such as QS-21, Quil-A, ISCOMs, ISCOMATRIXT™, emulsions such as MF59™, Montanide® ISA 51 and ISA 720, AS02 (QS21+squalene+MPL®), liposomes and liposomal formulations such as AS01, synthesized or specifically prepared microparticles and microcarriers such as bacteria-derived outer membrane vesicles (OMV) of N. gonorrheae, Chlamydia trachomatis and others, or chitosan particles, depot-forming agents, such as Pluronic® block co-polymers, specifically modified or prepared peptides, such as muramyl dipeptide, aminoalkyl glucosaminide 4-phosphates, such as RC529, or proteins, such as bacterial toxoids or toxin fragments. The doses of such other adjuvants can be determined using conventional dose ranging studies.

In embodiments, the inventive synthetic nanocarriers can be combined with an antigen different, similar or identical to those coupled to a nanocarrier (with or without adjuvant, utilizing or not utilizing another delivery vehicle) administered separately at a different time-point and/or at a different body location and/or by a different immunization route or with another antigen and/or adjuvant-carrying synthetic nanocarrier administered separately at a different time-point and/or at a different body location and/or by a different immunization route. In some embodiments, such antigen is another influenza antigen, for example, a neuraminidase, a surface antigen, a nucleocapsid protein, a matrix protein, a phosphoprotein, a fusion protein, a hemagglutinin, a hemagglutinin-neuraminidase, a glycoprotein capsular polysaccharides, a protein D, a M2 protein, or an antigenic fragment thereof, of an influenza virus.

In embodiments, the inventive dosage forms may comprise the recited synthetic nanocarriers and one or more conventional influenza vaccines to form a multivalent influenza vaccine. This may be accomplished by simply admixing a dispersion comprising the recited synthetic nanocarriers with a solution or dispersion that comprises a conventional influenza vaccine. In an embodiment, the inventive dosage forms comprise the recited synthetic nanocarriers and influenza antigen that is not coupled to the recited synthetic nanocarriers.

Populations of synthetic nanocarriers may be combined to form pharmaceutical dosage forms according to the present invention using traditional pharmaceutical mixing methods. These include liquid-liquid mixing in which two or more suspensions, each containing one or more subset of nanocarriers, are directly combined or are brought together via one or more vessels containing diluent. As synthetic nanocarriers may also be produced or stored in a powder form, dry powder-powder mixing could be performed as could the re-suspension of two or more powders in a common media. Depending on the properties of the nanocarriers and their interaction potentials, there may be advantages conferred to one or another route of mixing.

In some embodiments, inventive synthetic nanocarriers are manufactured under sterile conditions or are terminally sterilized. This can ensure that resulting composition are sterile and non-infectious, thus improving safety when compared to non-sterile compositions. This provides a valuable safety measure, especially when subjects receiving synthetic nanocarriers have immune defects, are suffering from infection, and/or are susceptible to infection. In some embodiments, inventive synthetic nanocarriers may be lyophilized and stored in suspension or as lyophilized powder depending on the formulation strategy for extended periods without losing activity.

The inventive compositions may be administered by a variety of routes of administration, including but not limited to intravenous, subcutaneous, pulmonary, intramuscular, intradermal, oral, intranasal, intramucosal, transmucosal, sublingual, rectal; ophthalmic, transdermal, transcutaneous or by a combination of these routes.

It is to be understood that the compositions of the invention can be made in any suitable manner, and the invention is in no way limited to compositions that can be produced using the methods described herein. Selection of an appropriate method may require attention to the properties of the particular moieties being associated.

The compositions and methods described herein can be used to induce, enhance, suppress, modulate, direct, or redirect an immune response. The compositions and methods described herein can be used in the diagnosis, prophylaxis and/or treatment of conditions such as human influenza infections or other related disorders and/or conditions.

### EXAMPLES

### Example 1

### Synthetic Nanocarriers with Covalently Coupled HA Polypeptide (Prophetic)

PLGA-R848 is prepared by reaction of PLGA polymer containing acid end group with R848 in the presence of coupling agent such as HBTU as follows:

A mixture of PLGA (Lakeshores Polymers, MW ˜5000, 7525DLG1A, acid number 0.7 mmol/g, 10 g, 7.0 mmol) and HBTU (5.3 g, 14 mmol) in anhydrous EtOAc (160 mL) is stirred at room temperature under argon for 50 minutes. Compound R848 (resiquimod, 2.2 g, 7 mmol) is added, followed by diisopropylethylamine (DIPEA) (5 mL, 28 mmol). The mixture is stirred at room temperature for 6 h and then at 50-55° C. overnight (about 16 h). After cooling, the mixture is diluted with EtOAc (200 mL) and washed with saturated NH₄Cl solution (2×40 mL), water (40 mL) and brine solution (40 mL). The solution is dried over Na₂SO₄ (20 g) and concentrated to a gel-like residue. Isopropyl alcohol (IPA) (300 mL) is then added and the polymer conjugate precipitated out of solution. The polymer is then washed with IPA (4×50 mL) to remove residual reagents and dried under vacuum at 35-40° C. for 3 days as a white powder (expected yields: 10.26 g, MW by GPC is 5200, R848 loading is 12% by HPLC).

PLA-PEG-N3 polymer is prepared by ring opening polymerization of HO-PEG-azide with dl-lactide in the presence of a catalyst such as Sn(Oct)2 as follows:

HO-PEG-CO2H (MW 3500, 1.33 g, 0.38 mmol) is treated with NH2-PEG3-N3 (MW 218.2, 0.1 g, 0.458 mmol) in the presence of DCC (MW 206, 0.117 g, 0.57 mmol) and NHS (MW 115, 0.066 g, 0.57 mmol) in dry DCM (10 mL) overnight. After filtration to remove insoluble byproduct (DCC-urea), the solution is concentrated and then diluted with ether to precipitate out the polymer, HO-PEG-N3 (1.17 g). After drying, HO-PEG-N3 (MW 3700, 1.17 g, 0.32 mmol) is mixed with dl-lactide (recrystallized from EtOAc, MW 144, 6.83 g, 47.4 mmol) and Na2SO4 (10 g) in a 100 mL flask. The solid mixture is dried under vacuum at 45 C overnight and dry toluene (30 mL) is added. The resulting suspension is heated to 110 C under argon and Sn(Oct)₂ (MW 405, 0.1 mL, 0.32 mmol) is added. The mixture is heated at reflux for 18 h and cooled to rt. The mixture is diluted with DCM (50 mL) and filtered. After concentration to an oily residue, MTBE (200 mL) is added to precipitate out the polymer which is washed once with 100 mL of 10% MeOH in MTBE and 50 mL of MTBE. After drying, PLA-PEG-N3 is obtained as a white foam (expected yield: 7.2 g, average MW: 23,700 by H NMR).

Synthetic nanocarriers (NC) made up of PLGA-R848, PLA-PEG-N3 (linker to polypeptide antigen) and ova peptide (T-helper antigen) are prepared via a double emulsion method wherein the ova peptide [ova (323-339), sequence: H-Ile-Ser-Gln-Ala-Val-His-Ala-Ala-His-Ala-Glu-Ile-Asn-Glu-Ala-Gly-Arg-NH2 (SEQ ID NO: 1), acetate salt, Lot#B06395, prepared by Bachem Biosciences, Inc.] is encapsulated in the NCs. To a suspension of the NCs (9.5 mg/mL in PBS (pH 7.4 buffer), 1.85 mL, containing about 4.4 mg (MW: 25,000; 0.00018 mmol, 1.0 eq) of PLA-PEG-N3) is added an HA polypeptide (Protein Sciences Corp. Meriden Conn.) containing a C-terminal alkyne linker (C-terminal glycine propargyl amide) (0.2-1 mM in PBS) with gentle stirring. A solution of Cu504 (100 mM in H2O, 0.1 mL) and a solution of copper (I) ligand, Tris(3-hydroxypropyltriazolylmethyl)amine (THPTA) (200 mM in H2O, 0.1 mL) are mixed and the resulting solution is added to the NC suspension. A solution of aminoguanidine hydrochloride salt (200 mM in H2O, 0.2 mL) is added, followed by a solution sodium ascorbate (200 mM in H2O, 0.2 mL). The resulting suspension is stirred at 4 C in dark for 18 h. The suspension is then diluted with PBS buffer (pH 7.4) to 5 mL and centrifuged to remove the supernatant. The residual NC pellets are washed with 2×5 mL PBS buffer. The washed NC-HA polypeptide conjugates are then re-suspended in 2 mL of PBS buffer and stored frozen until further analysis and biological tests.

### Example 2

### Synthetic Nanocarriers with Non-Covalently Coupled HA Polypeptide (Prophetic)

Synthetic nanocarriers having negative surface charges are prepared from polylactide, PLGA-R848 and ova peptide, as generally described in Example 1 above. The preparation takes place in the presence of long chain alkyl sulfate such as sodium dodecylsulfate or sulfonated polymer such as sodium polystyrene sulfonate using standard nanocarrier synthesis methods such as nanoprecipitation or double-emulsion evaporation method (such as that noted in Example 1 above). The negatively charged nanocarriers are then coated via ionic interactions with a positively charged HA polypeptide (Protein Sciences, Meriden Conn.) containing polylysine. The resulting non-covalently conjugated synthetic nanocarriers are then suspended in PBS buffer as described before for further analysis and biological tests.

### Example 3

### Synthetic Nanocarriers with Covalently Coupled HA Protein (Prophetic)

An HA glycoprotein (Protein Sciences, Meriden Conn.) is treated with succinic anhydride in the presence of base. The resulting succinic acid containing HA glycoprotein is then conjugated to a virus-like particle (VLP) such as an RNA bacteriophage, cowpea mosaic virus or Tobacco mosaic virus in the presence of EDC/NHS, adapting the techniques generally disclosed in U.S. Pat. No. 7,452,541 and/or US Published Patent application 2009/0238797. The resulting VLP-HA glycoprotein is then purified by dialysis and re-suspended in PBS solution for immunization study.

### Example 4

### Synthetic Nanocarriers with Covalently Coupled HA Polypeptide

In one embodiment of the present disclosure, the HA protein was conjugated to the synthetic nanocarrier via amide linker through NC containing surface carboxylic acid group as described below:

Synthetic nanocarriers (NC) made up of PLA-R848, PLA-PEG-CO2H with encapsulated ova peptide (prepared generally according to Example 1 above) were prepared by a double emulsion method. To a suspension of the NCs (14 mg/mL, 1 mL in pH=6 MES buffer, containing 0.16 umol, 1.0 eq of PLA-PEG-CO2H in the NCs) was added a freshly prepared solution of EDC (20 eq, 0.10 mL, 9 mg/mL in MES buffer) and NHS (40 eq, 0.10 mL, 10 mg/mL in MES buffer. The suspension was gently shaken at rt for 1 h. The suspension was diluted with PBS buffer (pH 7.4) to 3 mL and centrifuged to remove the supernatant containing excess EDC/NHS. The remaining NC pellets were washed once with 3 mL cold PBS buffer. The resulting activated NCs were then suspended in a solution of HA protein [H5A/Vietnam/1203/2004 protein obtained from Protein Sciences Corp. Meriden Conn., a full-length glycosylated recombinant protein of the strain A/Vietnam/1203/2004 (subtype H5N1), the HA protein was produced in insect cells using the baculovirus expression vector system and purified to >90% purity under conditions that were intended to preserve its biological and tertiary structure, the protein was characterized as MW 72 K; the solution was: 0.03 umol HA protein, 0.2 eq, 2 mg in 3 mL of PBS buffer]. The suspension was gently mixed at rt for 20 h. The suspension was centrifuged to remove the supernatant. The remaining NC pellets were washed twice with 2×3 mL PBS and re-suspended in 2 mL PBS (pH7.4) and stored frozen until further analysis and bioassay. Based on the amount of HA protein used, see Thorek et al. 2009 (cited above), the maximum loading of HA on the NCs was about 12.5% wt.

### Example 5

### In Vivo Testing of Synthetic Nanocarriers with Covalently Coupled HA Polypeptides

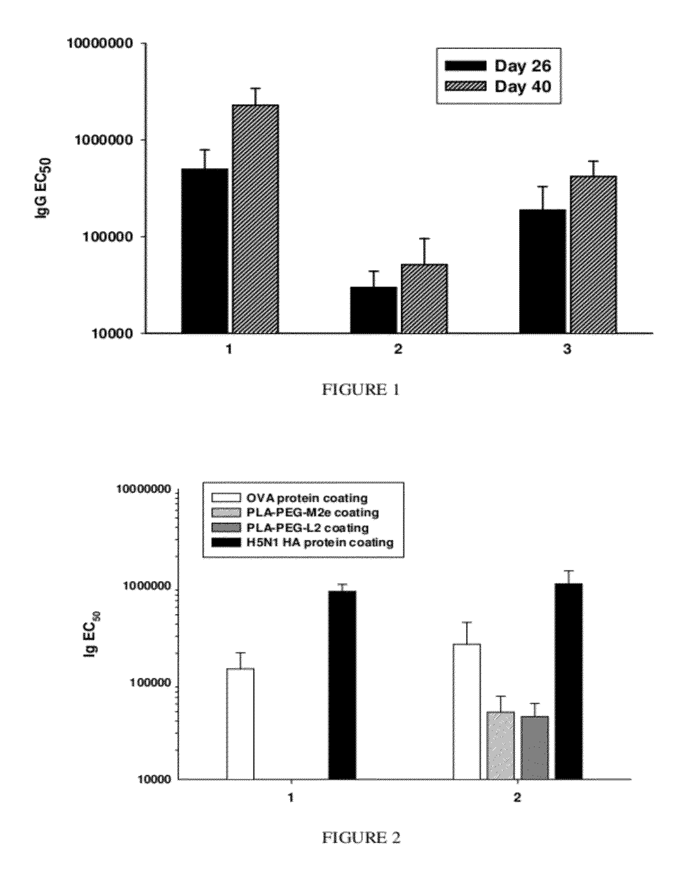

Synthetic nanocarriers were prepared according to Example 4. C57BL/6 mice were vaccinated using the synthetic nanocarriers (s.c., hind limbs, 60 μl total inoculation volume, 3 times with a 2-wk interval). Group 1: nanocarrier-HA protein conjugates (NC-HA), group 2: immunized with 1 μg of HA protein; group 3: immunized with 1 μg of HA in Imject alum (Thermo Scientific, w/w=1:1). Mice were bled at times indicated (Days 26 and 40 following initial vaccination) and anti-HA antibody titers determined by a standard ELISA against HA. Results are shown in FIG. 1.

Titers of anti-HA antibodies generated by NC-HA were nearly 50 times higher than those generated by immunization with 1 μg of HA protein and 4-5 times more when HA-alum mixture was used (FIG. 1). Notably, alum adjuvant is not used clinically for influenza immunization; therefore, comparing immunogenicity of NC-HA with purified HA demonstrates a true clinical value of applying NC-coupled HA for vaccination against influenza, while comparing immunogenicity of NC-HA to HA+alum underlines higher efficiency of NC than of other standard adjuvant.

### Example 6

### Preparation and Characterization of Nanocarrier Emulsions

### Preparation of Nanocarriers for Ovalbumin (Ova) Coating:

PLGA-R848, poly-D/L-lactide-co-glycolide, 4-amino-2-(ethoxymethyl)-α,α-dimethyl-1H-imidazo[4,5-c]quinoline-1-ethanol amide of approximately 7,000 Da made from PLGA of 3:1 lactide to glycolide ratio and having approximately 8.5% w/w conjugated resiquimod content was custom manufactured at Princeton Global Synthesis (300 George Patterson Drive #206, Bristol, Pa. 19007.)

PLA-PEG-Maleimide, block co-polymer consisting of a poly-D/L-lactide (PLA) block of approximately 22000 Da and a polyethylene glycol (PEG) block of approximately 2900 Da that is terminated by a maleimide functional group, was synthesized from commercial starting materials by generating the PLA block by ring-opening polymerization of dl-lactide with HO-PEG-Maleimide.

Polyvinyl alcohol PhEur, USP (85-89% hydrolyzed, viscosity of 3.4-4.6 mPa·s) was purchased from EMD Chemicals Inc. (480 South Democrat Road Gibbstown, N.J. 08027. Part Number 4-88).

Solutions were prepared as follows: Solution 1: 0.13N HCl in purified water. Solution 2: PLGA-R848 @ 50 mg/mL and PLA-PEG-Maleimide @ 50 mg/mL in dichloromethane was prepared by dissolving each polymer separately in dichloromethane at 100 mg/mL then combining 1 part PLGA-R848 solution to 1 part PLA-PEG-Maleimide solution. Solution 3: Polyvinyl alcohol @ 50 mg/mL in 100 mM in 100 mM phosphate buffer, pH 8. Solution 4: 70 mM phosphate buffer, pH 8.

A primary (W1/O) emulsion was first created using Solution 1 & Solution 2. Solution 1 (0.2 mL) and Solution 2 (1.0 mL) were combined in a small glass pressure tube and sonicated at 50% amplitude for 40 seconds using a Branson Digital Sonifier 250.

A secondary (W1/O/W2) emulsion was then formed by adding Solution 3 (2.0 mL) to the primary emulsion, vortexing to create a course dispersion, and then sonicating at 30% amplitude for 40 seconds using the Branson Digital Sonifier 250.

The secondary emulsion was added to an open 50 mL beaker containing 70 mM phosphate buffer solution (30 mL) and stirred at room temperature for 2 hours to allow the dichloromethane to evaporate and the nanocarriers to form in suspension. A portion of the suspended nanocarriers was washed by transferring the nanocarrier suspension to a centrifuge tube, spinning at 21,000 rcf for 45 minutes, removing the supernatant, and re-suspending the pellet in phosphate buffered saline. This washing procedure was repeated and then the pellet was re-suspended in phosphate buffered saline to achieve a nanocarrier suspension having a nominal concentration of 10 mg/mL on a polymer basis. The nanocarrier suspension was stored frozen at −20 C until further use.

TABLE 1
Nanocarrier characterization

| Effective Diameter (nm) | TLR Agonist, % w/w | T-cell agonist, % w/w |
|---|---|---|
| 208 | R848, 4.3 | None |

### Preparation of NC-OVA Conjugates:

Materials: (1) NC with PEG-MAL on the surface, prepared as described above; 6 mg/mL suspension in PBS buffer; (2) OVA protein (Ovalbumin from egg white): Worthington, Lot#POK12101, MW: 46000; (3) Traut's reagent (2-iminothiolane.HCl): MP Biomedical, Lot#8830KA, MW: 137.6; (4) pH 8 buffer (sodium phosphate, 20 mM with 0.5 mM EDTA); (5) pH 7 1×PBS buffer.

Procedure:

OVA protein (20 mg) was dissolved in 1 mL pH 8 buffer. A freshly made solution of Traut's reagent in pH 8 buffer (0.5 mL, 2 mg/mL) was added to the OVA protein solution. The resulting solution was stirred under argon in the dark for 1.5 h. The solution was diafiltered with MWCO 3K diafilter tube and washed with pH 8 buffer twice. The resulting modified OVA with thiol group was dissolved in 1 mL pH 8 buffer under argon. The NC suspension (4 mL, 6 mg/mL) was centrifuged to remove the supernatant. The modified OVA solution was then mixed with the NC pellets. The resulting suspension was stirred at rt under argon in the dark for 12 h. The NC suspension was then diluted to 10 mL with pH 7 PBS and centrifugated. The resulting NC was pellet washed with 2×10 mL pH 7 PBS. The NC-OVA conjugates were then resuspended in pH 7 PBS (ca. 6 mg/mL, 4 mL) stored at 4 C for further testing.

### Preparation of Nanocarriers for has Protein Coating:

Ovalbumin peptide 323-339 amide acetate salt, was purchased from Bachem Americas Inc. (3132 Kashiwa Street, Torrance Calif. 90505. Product code 4065609.) PLGA-R848, poly-D/L-lactide-co-glycolide, 4-amino-2-(ethoxymethyl)-α,α-dimethyl-1H-imidazo[4,5-c]quinoline-1-ethanol amide of approximately 7,000 Da made from PLGA of 3:1 lactide to glycolide ratio and having approximately 8.5% w/w conjugated resiquimod content was custom manufactured at Princeton Global Synthesis (300 George Patterson Drive #206, Bristol, Pa. 19007.) PLA-PEG-Maleimide, block co-polymer consisting of a poly-D/L-lactide (PLA) block of approximately 22000 Da and a polyethylene glycol (PEG) block of approximately 2900 Da that is terminated by a maleimide functional group, was synthesized from commercial starting materials by generating the PLA block by ring-opening polymerization of dl-lactide with HO-PEG-Maleimide. Polyvinyl alcohol PhEur, USP (85-89% hydrolyzed, viscosity of 3.4-4.6 mPa·s) was purchased from EMD Chemicals Inc. (480 South Democrat Road Gibbstown, N.J. 08027. Part Number 4-88).

Solutions were prepared as follows: Solution 1: Ovalbumin peptide 323-339 @ 20 mg/mL was prepared in 0.13N HCl at room temperature. Solution 2: PLGA-R848 @ 50 mg/mL and PLA-PEG-Maleimide @ 50 mg/mL in dichloromethane was prepared by dissolving each polymer separately in dichloromethane at 100 mg/mL then combining 1 part PLGA-R848 solution to 1 part PLA-PEG-Maleimide solution. Solution 3: Polyvinyl alcohol @ 50 mg/mL in 100 mM in 100 mM phosphate buffer, pH 8. Solution 4: 70 mM phosphate buffer, pH 8.

A primary (W1/O) emulsion was first created using Solution 1 & Solution 2. Solution 1 (0.2 mL) and Solution 2 (1.0 mL) were combined in a small glass pressure tube and sonicated at 50% amplitude for 40 seconds using a Branson Digital Sonifier 250.

A secondary (W1/O/W2) emulsion was then formed by adding Solution 3 (2.0 mL) to the primary emulsion, vortexing to create a course dispersion, and then sonicating at 30% amplitude for 40 seconds using the Branson Digital Sonifier 250.

The secondary emulsion was added to an open 50 mL beaker containing 70 mM phosphate buffer solution (30 mL) and stirred at room temperature for 2 hours to allow the dichloromethane to evaporate and the nanocarriers to form in suspension. A portion of the suspended nanocarriers was washed by transferring the nanocarrier suspension to a centrifuge tube, spinning at 21,000 rcf for 45 minutes, removing the supernatant, and re-suspending the pellet in phosphate buffered saline. This washing procedure was repeated and then the pellet was re-suspended in phosphate buffered saline to achieve a nanocarrier suspension having a nominal concentration of 10 mg/mL on a polymer basis. The nanocarrier suspension was stored frozen at −20 C until further use.

TABLE 2
Nanocarrier characterization

| Effective Diameter | | |
|---|---|---|
| (nm) | TLR Agonist, % w/w | T-cell agonist, % w/w |
| 216 | R848, 3.6 | Ova peptide 323-339, 2.0 |

### Preparation of NC-HA5 Protein Conjugates:

Materials: (1) NC with PEG-MAL on the surface, prepared as described above; 6.7 mg/mL suspension in PBS buffer; (2) HAS protein: Recombinant Hemagglutinin, A/Vietnam/1203/2004, MW: 72000, supplied as a solution in pH 7 PBS-tween buffer (0.55 mg/mL); (3) Traut's reagent (2-iminothiolane.HCl): MP Biomedical, Lot#8830KA, MW: 137.6; (4) pH 8 buffer (sodium phosphate, 20 mM with 0.5 mM EDTA); (5) pH 7 1×PBS buffer.

Procedure: HAS protein (0.21 g in 0.38 mL pH 7.1 PBS-tween buffer) was diluted to 0.5 mL with pH 8 buffer. A freshly made solution of Traut's reagent in pH 8 buffer (0.02 mL, 2 mg/mL) was added to the HAS protein solution. The resulting solution was stirred under argon in the dark for 1.5 h. The solution was diafiltered with MWCO 3K diafilter tube and washed with pH 8 buffer twice. The resulting modified HAS protein with thiol group was dissolved in 0.5 mL pH 8 buffer under argon. The NC suspension (3 mL, 6.7 mg/mL) was centrifugated to remove the supernatant. The modified HAS solution was then mixed with the NC pellets. The resulting suspension was stirred at rt under argon in the dark for 12 h. The NC suspension was then diluted to 10 mL with pH 7 PBS and centrifuged. The resulting NC was pellet washed with 2×10 mL pH 7 PBS. The NC-HA5 conjugates were then resuspended in pH 7 PBS (ca. 6 mg/mL, 3 mL) stored at 4 C for further testing.

Preparation of Nanocarriers for L2, M2e, or M2e-L2 Coating:

Materials: Ovalbumin peptide 323-339 amide acetate salt, was purchased from Bachem Americas Inc. (3132 Kashiwa Street, Torrance Calif. 90505. Product code 4065609.) PLGA-R848, poly-D/L-lactide-co-glycolide, 4-amino-2-(ethoxymethyl)-α,α-dimethyl-1H-imidazo[4,5-c]quinoline-1-ethanol amide of approximately 7,000 Da made from PLGA of 3:1 lactide to glycolide ratio and having approximately 8.5% w/w conjugated resiquimod content was custom manufactured at Princeton Global Synthesis (300 George Patterson Drive #206, Bristol, Pa. 19007.) PLA-PEG-C6-N3, block co-polymer consisting of a poly-D/L-lactide (PLA) block of approximately 23000 Da and a polyethylene glycol (PEG) block of approximately 2000 Da that is terminated by an amide-conjugated C6H12 linker to an azide, was synthesized by conjugating HO-PEG-COOH to an amino-C6H12-azide and then generating the PLA block by ring-opening polymerization of the resulting HO-PEG-C6-N3 with dl-lactide. Polyvinyl alcohol PhEur, USP (85-89% hydrolyzed, viscosity of 3.4-4.6 mPa·s) was purchased from EMD Chemicals Inc. (480 South Democrat Road Gibbstown, N.J. 08027. Part Number 4-88).

Method: Solutions were prepared as follows: Solution 1: Ovalbumin peptide 323-339 @ 20 mg/mL was prepared in phosphate buffered saline at room temperature. Solution 2: PLGA-R848 @ 50 mg/mL and PLA-PEG-C6-N3 @ 50 mg/mL in dichloromethane was prepared by dissolving each separately at 100 mg/mL in dichloromethane then combining in equal parts by volume. Solution 3: Polyvinyl alcohol @ 50 mg/mL in 100 mM in 100 mM phosphate buffer, pH 8. Solution 4: 70 mM phosphate buffer, pH 8.

A primary (W1/O) emulsion was first created using Solution 1 & Solution 2. Solution 1 (0.2 mL) and Solution 2 (1.0 mL) were combined in a small glass pressure tube and sonicated at 50% amplitude for 40 seconds using a Branson Digital Sonifier 250. A secondary (W1/O/W2) emulsion was then formed by adding Solution 3 (2.0 mL) to the primary emulsion, vortexing to create a course dispersion, and then sonicating at 30% amplitude for 40 seconds using the Branson Digital Sonifier 250. The secondary emulsion was added to an open 50 mL beaker containing 70 mM phosphate buffer solution (30 mL) and stirred at room temperature for 2 hours to allow the dichloromethane to evaporate and the nanocarriers to form in suspension. A portion of the suspended nanocarriers was washed by transferring the nanocarrier suspension to a centrifuge tube, spinning at 21,000 rcf for 45 minutes, removing the supernatant, and re-suspending the pellet in phosphate buffered saline. This washing procedure was repeated and then the pellet was re-suspended in phosphate buffered saline to achieve a nanocarrier suspension having a nominal concentration of 10 mg/mL on a polymer basis. Two identical batches were created and then combined to form a single homogenous suspension at which was stored frozen at −20 C until further use.

TABLE 3
Azide-functionalized nanocarrier characterization

| Effective Diameter | | |
|---|---|---|
| (nm) | TLR Agonist, % w/w | Antigen, % w/w |
| 209 | R848, 4.2 | Ova 323-339 peptide, 2.4 |

Preparation of NC-M2e-L2 Conjugates:

Materials: (1) Nanocarriers with surface PEG-C6-N3 containing PLGA-R848 and Ova-peptide, prepared as described above, 7 mg/mL suspension in PBS. (2) HPV16 L2 peptide modified with an alkyne linker attached to C-terminal Lys amino group; Bachem Americas, Inc, Lot B06055, MW 2595, TFA salt Sequence: H-Ala-Thr-Gln-Leu-Tyr-Lys-Thr-Cys-Lys-Gln-Ala-Gly-Thr-Cys-Pro-Pro-Asp-Ile-Ile-Pro-Lys-Val-Lys(5-hexynoyl)-NH2 (with Cys-Cys disulfide bond) (SEQ ID NO: 2), (3) M2e peptide modified with an alkyne linker attached to C-terminal Gly; CS Bio Co, Catalog No. CS4956, Lot: H308, MW 2650, TFA salt; Sequence: H-Met-Ser-Leu-Leu-Thr-Glu-Val-Glu-Thr-Pro-Thr-Arg-Asn-Glu-Trp-Glu-Cys-Arg-Cys-Ser-Asp-Gly-Gly-NHCH2CCH (SEQ ID NO: 3), (4) Catalysts: CuSO4, 100 mM in DI water; THPTA ligand, 200 mM in DI water; sodium ascorbate, 200 mM in DI water freshly prepared. (5) pH 7.4 PBS buffer. In embodiments, sequence: H-Met-Ser-Leu-Leu-Thr-Glu-Val-Glu-Thr-Pro-Ile-Arg-Asn-Glu-Trp-Glu-Cys-Arg-Cys-Ser-Asp-Gly-Gly-NHCH2CCH (SEQ ID NO: 4) could instead be used.

Procedures: The NC suspension (7 mg/mL, 2 mL) was concentrated to ca. 0.5 mL in volume by centrifugation. A mixture of L2 peptide (5 mg) and M2e peptide (5 mg) in 1 mL PBS buffer was added. A pre-mixed solution of 0.2 mL of CuSO4 (100 mM) and 0.2 mL of THPTA ligand (200 mM) was added, followed by 0.4 mL of sodium ascorbate (200 mM). The resulting light yellow suspension was stirred in dark at ambient room temperature for 18 h. The suspension was then diluted with PBS buffer to 10 mL and centrifugated to remove the supernatant. The NC-M2e-L2 conjugates were further pellet washed twice with 10 mL PBS buffer and resuspended in pH 7.4 buffer at final concentration of ca. 6 mg/mL (ca. 2 mL) and stored at 4 C for further testing.

### Example 7

### In Vivo Testing of Synthetic Nanocarriers with Covalently Coupled HA

Polypeptides in the Presence of Nanocarriers with Covalently Coupled Proteins or Peptides

Synthetic nanocarriers were prepared according to above examples.

C57BL/6 mice were vaccinated using the synthetic nanocarriers (s.c., hind limbs, 60 μL total inoculation volume, 2 times with a 3-week interval). Group 1: immunized with nanocarrier-HA protein conjugates (NC-HA) and nanocarrier-ovalbumin protein conjugates (NC-OVA). Group 2: immunized with NC-HA, NC-OVA, and nanocarrier-M2e peptide-L2 peptide conjugates (NC-M2e-L2; influenza M2e peptide, HPV L2 peptide). Mice were bled at day 33 and anti-HA, anti-OVA, anti-M2e peptide, and anti-L2 peptide antibody titers determined by a standard ELISA against HA protein, OVA protein, M2e peptide, or L2 peptide. Results are shown in FIG. 2.

Titers of anti-HA antibodies generated by mice immunized with both NC-HA and NC-OVA were comparable to those generated by mice immunized with NC-HA alone (FIGS. 1 and 2). These mice also generated antibodies to ovalbumin (FIG. 2). Titers of anti-HA antibodies generated by mice immunized with the three nanocarriers (NC-HA, NC-OVA, and NC-M2e-L2) were comparable to those generated by mice immunized with NC-HA alone or both NC-HA and NC-OVA (FIGS. 1 and 2).

### REFERENCES

Black, M., Trent A., Tirrel M., and Olive, C. Advances in the design and delivery of peptide subunit vaccines with a focus on Toll-like receptor agonists. Expert Rev. Vaccines 2010; 9:157-173.
Caton, A. J., Brownlee, G. G., Yewdell, J. M. and Gerhard, W. The antigenic structure of the influenza virus A/PR/8/34 hemagglutinin (H1 subtype). Cell. 1982; 31:417-427.
Chakrabartty A., Kortemme T., Baldwin R. L. Helix propensities of the amino acids measured in alanine-based peptides without helix-stabilizing side-chain interactions. Protein Sci. 1994; 3:843-852.
Cross K. J., Langley W. A., Russell R. J., et al. Composition and functions of the influenza fusion peptide. Protein Pept. Lett. 2009; 16:766-778.
Ellebedy A. H., Webby R. J. Influenza vaccines. Vaccine. 2009; 27 Suppl. 4:D65-8.
Kaverin N. V., Rudneva I. A., Ilyushina N. A., et al. Structure of antigenic sites on the haemagglutinin molecule of H5 avian influenza virus and phenotypic variation of escape mutants. J Gen Virol. 2002; 83:2497-2505.
Mozdzanowska K., Zharikova D., Cudic M., et al. Roles of adjuvant and route of vaccination in antibody response and protection engendered by a synthetic matrix protein 2-based influenza A virus vaccine in the mouse. Virol. J. 2007; 4:118.
Purcell, A. W., Zeng, W., Mifsud, N. A., et al. Dissecting the Role of Peptides in the Immune Response Theory, Practice and the Application to Vaccine Design. J. Peptide Sci. 2003; 9: 255-281.
Roose K., Fiers W., Saelens X. Pandemic preparedness: toward a universal influenza vaccine. Drug News Perspect. 2009; 22:80-92.
Sui J., Hwang W. C., Perez S., et al. Structural and functional bases for broad-spectrum neutralization of avian and human influenza A viruses. Nat Struct Mol Biol. 2009; 16:265-273.
Throsby M., van den Brink E., Jongeneelen M., et al. Heterosubtypic neutralizing monoclonal antibodies cross-protective against H5N1 and H1N1 recovered from human IgM+memory B cells. PLoS One. 2008; 3:e3942.
Tsuchiya, E., Sugawara, K., Hongo, S., Matsuzaki, Y., Muraki, Y., Li, Z.-N. and Nakamura, K. Antigenic structure of the haemagglutinin of human influenza A/H2N2 virus. Journal of General Virology 2001; 82:2475-2484.
Vu Hong, Stanislav I. Presolski, Celia Ma, M. G. Finn; Analysis and Optimization of Copper-Catalyzed Azide-Alkyne Cycloaddition for Bioconjugation; Angewandte Chemie International Edition; 2009, 48: 9879-9883.
Wiley, D. C., Wilson, I. A., Skehel, J. J. Structural identification of the antibody-binding sites of Hong Kong influenza haemagglutinin and their involvement in antigenic variation. Nature. 1981; 289:373-378.

1. A dosage form comprising:
synthetic nanocarriers that are coupled to polypeptides obtained or derived from human influenza A virus hemagglutinin.

2. The dosage form of claim 1, wherein the polypeptides are glycosylated.

3. The dosage form of claim 1, wherein the polypeptides comprise an entire human influenza A virus hemagglutinin.

4. The dosage form of claim 1, wherein the polypeptides comprise a fragment of human influenza A virus hemagglutinin.

5. The dosage form of claim 1, wherein the polypeptides are obtained or derived from an HA1 subunit of human influenza A virus hemagglutinin.

6. The dosage form of claim 5, wherein the polypeptides comprise an entire HA1 subunit of human influenza A virus hemagglutinin, or a fragment thereof.

7. (canceled)

8. The dosage form of claim 1, wherein the polypeptides are obtained or derived from an HA2 subunit of human influenza A virus hemagglutinin.

9. The dosage form of claim 8, wherein the polypeptides comprise an entire HA2 subunit of human influenza A virus hemagglutinin, or a fragment thereof.

10. (canceled)

11. The dosage form of claim 1, wherein the synthetic nanocarriers are further coupled to one or more adjuvants.

12-13. (canceled)

14. The dosage form of claim 1, wherein the synthetic nanocarriers comprise lipid-based nanoparticles, polymeric nanoparticles, metallic nanoparticles, surfactant-based emulsions, dendrimers, buckyballs, nanowires, virus-like particles, peptide or protein-based particles, lipid-polymer nanoparticles, spheroidal nanoparticles, cubic nanoparticles, pyramidal nanoparticles, oblong nanoparticles, cylindrical nanoparticles, or toroidal nanoparticles, and, optionally wherein the synthetic nanocarriers comprise poly(lactic acid)-polyethyleneglycol copolymer, poly(glycolic acid)-polyethyleneglycol copolymer, or poly(lactic-co-glycolic acid)-polyethyleneglycol copolymer.

15. (canceled)

16. The dosage form of claim 1, wherein the synthetic nanocarriers are further coupled to one or more T-helper antigens.

17. (canceled)

18. The dosage form of claim 1, further comprising influenza antigen that is not coupled to the synthetic nanocarriers.

19. The dosage form of claim 1, wherein at least a portion of the polypeptides obtained or derived from human influenza A virus hemagglutinin are coupled to a surface of the synthetic nanocarriers.

20. The dosage form of claim 1, wherein the synthetic nanocarriers are covalently coupled to polypeptides obtained or derived from human influenza A virus hemagglutinin.

21. The dosage form of claim 1, wherein the synthetic nanocarriers are non-covalently coupled to polypeptides obtained or derived from human influenza A virus hemagglutinin.

22. (canceled)

23. A method comprising administering the dosage form of claim 1 to a subject.

24-27. (canceled)

28. A method comprising:
providing synthetic nanocarriers; and
coupling polypeptides that are obtained or derived from human influenza A virus hemagglutinin to the synthetic nanocarriers.

29. The method of claim 28, wherein coupling comprises covalently coupling the polypeptides to the synthetic nanocarriers.

30. A composition, dosage form or vaccine obtained, or obtainable, by a method as defined in claim 28.

31. A process for producing a composition, dosage form or vaccine comprising the steps of:
providing synthetic nanocarriers; and
coupling polypeptides that are obtained or derived from human influenza A virus hemagglutinin to the synthetic nanocarriers.

32-37. (canceled)
